(19) United States
(12) Patent Application Publication
(10) Pub. No.: US 2026/0231299 A1
(43) Pub. Date: Aug. 6, 2026

(54) ELECTRIC GOGGLES FOR PREVENTING FOGGING OF THE LENS

(71) Applicant: KIMPEX INC., Drummondville (CA)

(72) Inventors: Frédéric RENÉ-LAFOREST, Drummondville (CA); Jean-Simon LÉVESQUE, Drummondville (CA); Stéphane DION, Drummondville (CA); Martin TANGUAY, Drummondville (CA); Stanley BEAL, Drummondville (CA); Mathis BUSSIERE, Drummondville (CA)

(21) Appl. No.: 19/532,447

(22) Filed: Feb. 6, 2026

Related U.S. Application Data
(60) Provisional application No. 63/754,848, filed on Feb. 6, 2025.

(51) Int. Cl. H05B 3/84; A61F 9/02; G02B 1/16; G02B 1/18; H05B 3/00

(52) U.S. Cl. CPC H05B 3/84; A61F 9/027; A61F 9/029; G02B 1/16; G02B 1/18; H05B 3/0004

(57) ABSTRACT

A pair of goggles is provided and includes a frame defining a closed perimeter and an opening, a strap assembly coupled to the frame and a lens assembly removably connectable to the frame to cover the opening. The lens assembly includes a lens frame defining a lens opening, and a lens connectable to the lens frame to cover the lens opening. The goggles also include a heating assembly configured to generate and provide heat to the lens assembly. The heating assembly includes conductive elements provided on the lens and configured to enable an electrical current to travel therebetween, the conductive elements being provided on opposite sides of the lens such that, upon connecting the lens to the lens frame, the conductive elements are located proximate respective lateral edges of the lens frame.

### TECHNICAL FIELD

The technical field generally relates to electric goggles suitable for use with a helmet, and more specifically relates to electric goggles provided with a heating assembly for preventing fogging of the lens.

### BACKGROUND

Frost or condensation formation on the lenses of goggles used for practicing sports and outdoor activities (e.g., during winter) is undesirable. Known goggles are provided with a heated lens to prevent or reduce frost or condensation on the lens. A known design of the heated lenses includes providing a thin conductive film to a surface of the lens, then having an electric current travel across the conductive film. Many of the commonly available transparent conductive films rely upon inorganic compounds to provide the necessary conductivity. However, one of the challenges is providing a thin film with a conductive material that has both sufficient conductivity and transparency. Patterns or opaque layers including embedded metal wires affect visual performance, while insufficient conductivity limits heating speed and range.

In view of the above, many challenges still exist in the field of electric goggles.

### SUMMARY

According to an aspect of the present disclosure, a pair of goggles to be worn in combination with a helmet to selectively cover a front opening of the helmet is provided. The pair of goggles include a frame having a top frame edge, a bottom frame edge and opposite lateral frame edges together defining a closed perimeter and an opening; a strap assembly comprising at least one strap clip coupled to the frame and a strap connectable to the strap clip, the strap being adapted to surround the helmet or be connected thereto; a lens assembly removably connectable to the frame to cover the opening. The lens assembly comprising a lens frame having a top lens edge, a bottom lens edge and opposite lateral lens edges defining a lens opening; and a lens connectable to the lens frame to cover the lens opening. The goggles further comprising a heating assembly configured to generate and provide heat to the lens assembly. The heating assembly has conductive elements provided on the lens and configured to enable an electrical current to travel therebetween, the conductive elements being provided on opposite sides of the lens such that, upon connecting the lens to the lens frame, the conductive elements are located proximate respective lateral lens edges of the lens frame.

According to a possible embodiment, the conductive elements include two conductive elements spaced from one another across the lens, each conductive element extending along generally an entire height of the opposite sides of the lens such that, upon connecting the lens to the lens frame, the conductive elements extend between the top lens edge and the bottom lens edge.

According to a possible embodiment, the conductive elements are integrated with the lens or are printed on the lens.

According to a possible embodiment, the conductive elements are printed on the lens using conductive ink containing silver, carbon particles or both.

According to a possible embodiment, the heating assembly comprises a resistive structure extending across the lens between the conductive elements, the resistive structure being configured to assist in carrying the electrical current between the conductive elements.

According to a possible embodiment, the resistive structure comprises a transparent conductive film applied to a surface of the lens.

According to a possible embodiment, the transparent conductive film comprises a substrate overcoated with a nanowire layer and a conductive layer.

According to a possible embodiment, the conductive layer comprises at least one of carbon nanotubes, a copper micromesh, silver nanowires and a binder.

According to a possible embodiment, the resistive structure comprises a conductive film compound including indium tin oxide.

According to a possible embodiment, the heating assembly comprises an electric system operable to provide the electric current to the lens. The electric system comprising a power source; electrical wiring housed within the frame; connection pins provided on the frame and operatively connectable to the power source via the electrical wiring, wherein the conductive elements are adapted to engage with the connection pins to establish an electrical connection therewith upon connecting the lens assembly with the frame.

According to a possible embodiment, the connection pins comprise a pair of internal connection pins provided proximate respective lateral frame edges, each internal connection pin being adapted to engage with respective conductive elements upon connecting the lens assembly with the frame; external connection pins electrically coupled to the internal connection pins via the electrical wiring, the external connection pins being configured to engage and connect with the power source.

According to a possible embodiment, the frame comprises outriggers extending outwardly on opposite sides thereof, and wherein the external connection pins are provided on the outriggers.

According to a possible embodiment, the strap assembly comprises a pair of strap clips connectable to respective sides of the frame, each strap clip comprising electrical components configured to engage and connect with the external connection pins.

According to a possible embodiment, the power source is connectable to the electrical components of the strap clip, and wherein the strap clip is adapted to be connected to the frame to have the electrical components engage and connect with the external connection pins, thereby establishing an electrical connection between the power source and the external connection pins.

According to a possible embodiment, the electric system defines a closed-loop electric circuit.

According to a possible embodiment, the lens comprises one or more electrical conductors spaced from the conductive elements and configured to bias the electrical current travelling across the lens towards the electrical conductors.

According to a possible embodiment, the electrical conductors include metallic components distributed on the lens between the conductive elements and/or a mechanical break in the resistive structure of the heating system such that the electrical current is biased over and/or under the mechanical break.

According to a possible embodiment, the mechanical break corresponds to a kiss cut defined across the lens.

According to another aspect, a lens assembly for a pair of electric goggles provided with a heating assembly is provided. The lens assembly comprising a lens frame having a top lens edge, a bottom lens edge and opposite lateral lens edges defining a lens opening; and a lens connectable to the lens frame to cover the lens opening, the lens comprising conductive elements spaced across a surface of the lens and configured to enable an electrical current to travel therebetween, the conductive elements being provided on opposite sides of the lens such that, upon connecting the lens to the lens frame, the conductive elements are located proximate respective lateral lens edges of the lens frame.

According to another aspect, a pair of electrical goggles is provided. The pair of electrical goggles comprising a three-part assembly including a rigid frame; an inner component removably connected to a first side of the rigid frame; an outer component removably connected to a second side of the rigid frame, opposite the first side. The pair of electrical goggles also having According to a possible embodiment, a heating assembly configured to generate and provide heat to at least one of the inner component and the outer component. The heating assembly comprising a pair of conductive elements provided on the at least one of the inner component and the outer component and configured to enable an electrical current to travel therebetween, the conductive elements being provided on opposite ends of the at least one of the inner component and the outer component such that the electrical current travels across a distance of the at least one of the inner component and the outer component, the distance corresponding to a greater one between a length and a height of the at least one of the inner component and the outer component.

### BRIEF DESCRIPTION OF THE DRAWINGS

FIG. 1 is a perspective view of goggles strapped to a helmet, according to an embodiment.

FIG. 2 is a perspective view of the goggles shown in FIG. 1, showing a lens assembly coupled to a frame, according to an embodiment.

FIG. 3 is an exploded view of the goggles shown in FIG. 2, showing components of the lens assembly and of the frame, according to an embodiment.

FIG. 4 is an enlarged view of the frame shown in FIG. 3, showing a frame body, an internal frame component and a frame cover, according to an embodiment.

FIG. 4A is an enlarged view of a part of FIG. 4, showing outrigger covers provided with pin openings, according to an embodiment.

FIG. 4B is an enlarged view of a part of FIG. 4, showing a vent actuator operable for actuation of a ventilation system, according to an embodiment.

FIG. 5 is a front view of the frame body shown in FIG. 4, showing the ventilation system proximate a top edge thereof, according to an embodiment.

FIG. 6 is a rear view of the frame body shown in FIG. 4, showing slots define about a periphery of the frame body to receive magnets, according to an embodiment.

FIG. 7 is an enlarged view of the lens assembly shown in FIG. 3, showing a lens frame, an internal lens and an external lens, according to an embodiment.

FIG. 8 is a perspective view of the goggles shown in FIG. 2, showing the lens assembly disconnected from the frame, according to an embodiment.

FIG. 9 is a perspective view of the frame shown in FIG. 4, showing magnets of a magnetic connection assembly for insertion in respective magnet slots, according to an embodiment.

FIG. 10 is a rear perspective view of the lens assembly shown in FIG. 8, showing magnets of the magnetic connection assembly for insertion in respective magnet slots, according to an embodiment.

FIG. 11A is a perspective view of a magnet of the magnetic connection assembly, according to an embodiment.

FIG. 11B is a side view of the magnet shown in FIG. 11A, showing a trapezoidal and/or truncated pyramid shape of the magnet, according to an embodiment.

FIG. 12 is a perspective view of the frame body shown in FIG. 9, showing connection pins provided about a periphery thereof, according to an embodiment.

FIG. 13 is a front view of the lens assembly according to an embodiment, showing conductive elements positioned at opposite lateral ends.

FIG. 13A is an enlarged view of a part of FIG. 13, showing a structure of a transparent conductive film, according to an embodiment.

FIG. 14 is a front view of the goggles shown in FIG. 2, showing an electrical circuit, including a power source and straps for closing the electrical circuit, according to an embodiment.

FIG. 15 is a front view of the goggles according to another embodiment, showing an electrical device coupled to the electrical circuit.

FIGS. 16A to 18B are schematic view of the goggles shown in FIG. 2, showing temperature distributions across the lens during operation of the heating system at different operational specifications, according to some embodiments.

FIGS. 19A to 22B are schematic view of prior art goggles, showing temperature distributions across the lens during operation of the heating system at different operational specifications, according to some embodiments.

FIG. 23 is a front view of an alternate embodiment of the internal lens of the lens assembly, showing lateral bus bars with electrodes proximate a bottom thereof.

FIG. 24 is a perspective exploded view of the internal lens shown in FIG. 23, showing a stackup assembly forming a structure of the internal lens according to the alternate embodiment.

### DETAILED DESCRIPTION

As will be explained below in relation to various implementations, the present disclosure describes systems, devices and associated methods and processes for heated lenses, such as the lens on a pair of goggles. The goggles can correspond to a pair of electric goggles useable in cold-weather environments, for example, when riding a motorcycle or snowmobile, or when doing certain activities such as skiing or walking in the outdoors (e.g., trekking, snowshoeing, etc.). While the present disclosure describes electric goggles for use in cold weather and/or during winter, it should be noted that the electric goggles can be used in other environments and for other purposes than cold-weather sports. For instance, the goggles can be used in mild and/or hot weather to protect the wearer's face from dust, sand, debris and/or harsh sunlight, among others.

The present disclosure more particularly relates to electric goggles (hereafter simply “goggles”) having a frame and a lens assembly. The lens assembly is removably connectable to the frame of the goggles via an attachment system which can include an array of magnets, for example. The lens assembly can thus be selectively connected and removed from the frame. The lens assembly includes a heating system configured to provide energy (e.g., heat) to the lens of the goggles. The heat can be useful to remove and/or prevent snow, ice, condensation and fog from accumulating on the lens. It should therefore be appreciated that the heating system can provide an improved field of view and overall vision to the user by assisting in maintaining the surface of the lens generally clear.

In some implementations, the heating system includes a heating film coupled to the lens of the goggles and adapted to provide heat thereto. The heating film is generally transparent and has sufficient light transparency (e.g., visible light transmission (VLT)) to avoid hindering visibility of the user. For instance, the transparent electric heating film can have over 80% light transparency or “VLT”, such as over 90% VLT. The heating film corresponds to an electric heating film and is thus powered via an electrical source of power. For instance, the goggles can be battery-powered and/or adapted to be electrically connected to a vehicle, such as a snowmobile, via a power cable. As will be described below, the heating system includes electrodes, printed electrical circuits and/or electrical wires integrated to the structure of the goggles and configured to provide an electrical current for heating the electric film (i.e., for heating the surface of the lens).

In some embodiments, the electric film includes single-wall carbon nanotubes, a metal micromesh (e.g., copper micromesh) and/or silver nanowires adapted to improve electrical conductivity across the surface of the lens, thereby improving heat transfer across the lens. In addition, the lens can be customized to improve heat distribution and prevent, or at least reduce, cold spots on the lens. For instance, manufacturing processes can be implemented to lead or bias the electrical current to specific portions of the lens to provide heat to those portions. The manufacturing processes can include kiss cutting (e.g., surface cutting) the lens proximate the bridge of the nose and/or above the eyes to lead electrical current towards lower and upper portions of the lens, respectively, for heating purposes.

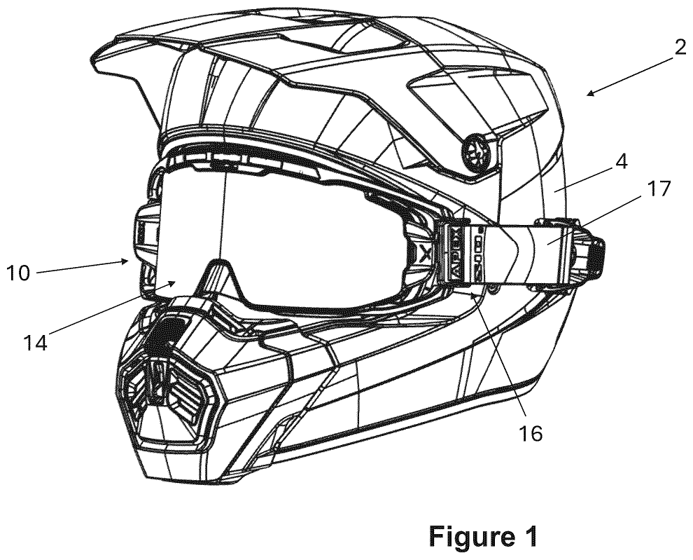

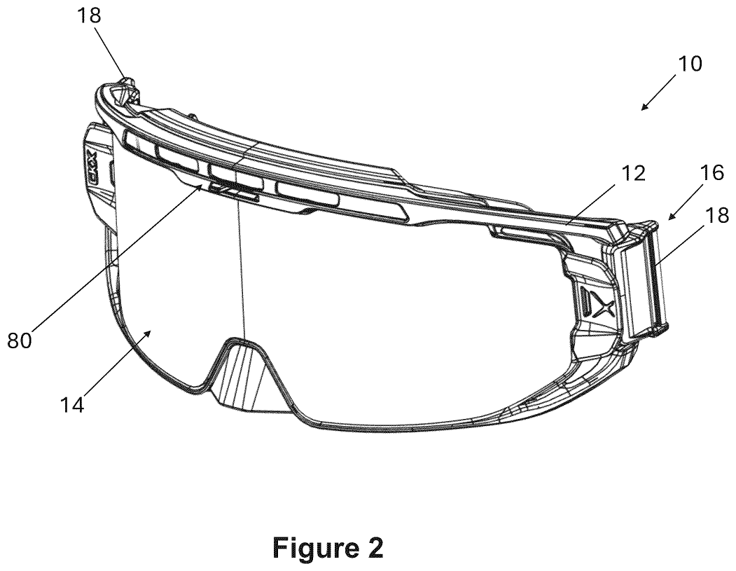

With reference to FIGS. 1 to 3, a pair of goggles 10 according to an embodiment is shown. The goggles 10 include a frame 12 and a lens assembly 14 connectable to the frame 12. The goggles 10 also include a strap assembly 16 configured to enable connection of a strap 17 (shown in FIGS. 1, 14 and 15), such as a resilient band, for wearing the goggles (e.g., around a user/wearer's head or around a helmet 2). In this embodiment, the strap assembly 16 includes a pair of strap clips 18 mountable to opposite sides of the frame 12 to enable connection of the strap to the frame. This type of strap assembly is well known in the art and will not be detailed further. The goggles 10 further include a heating assembly 100 configured to generate and provide heat to the lens assembly 14 for removing and/or preventing snow, ice, condensation and fog from accumulating on the lens assembly.

As described herein, the goggles 10 are configured to be fitted or “used” in combination with a helmet 2. It should be understood that the helmet 2 includes a protective helmet shell 4 defining a cavity shaped and configured to receive a user's head. The helmet shell 4 traditionally has a front opening communicating with the cavity to allow the user to see. The goggles 10 are configured to engage the front opening of the helmet shell 4, thereby protecting the user's face, eyes, etc., when wearing the goggles. As will be described below, the goggles 10 can be adapted to define a seal with the user's face and/or with a periphery of the front opening of the helmet shell. This configuration can improve comfort and performance of the goggles.

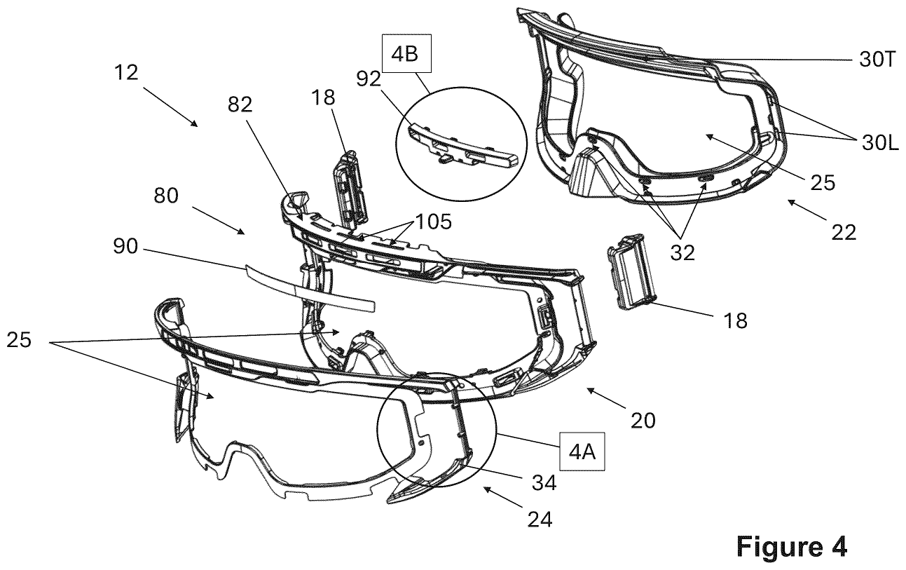

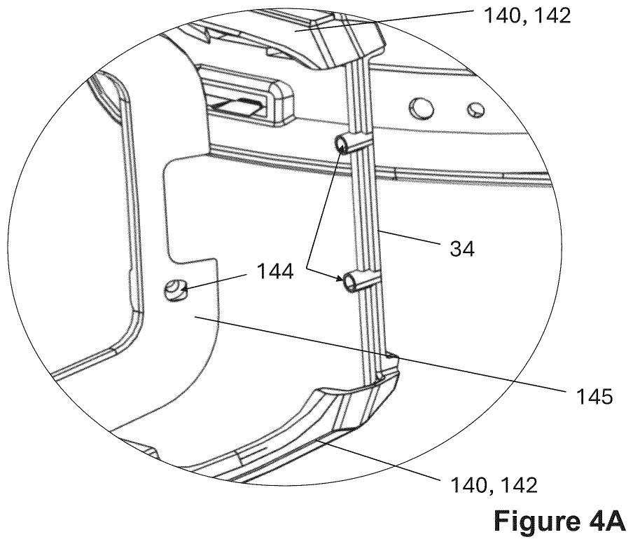

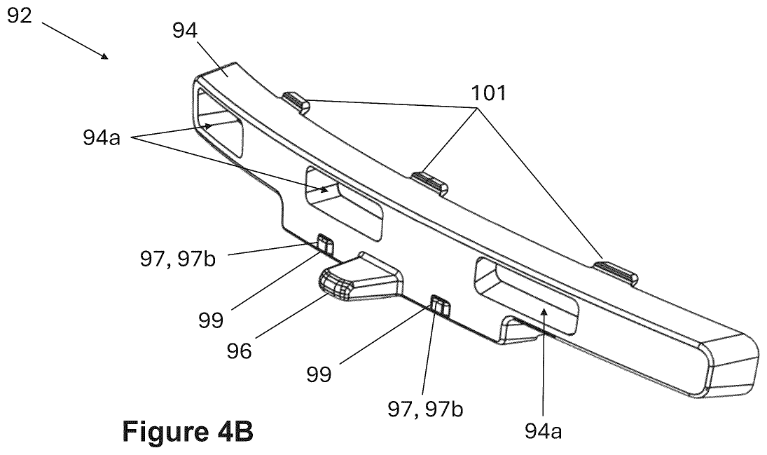

Now referring to FIG. 4, in addition to FIGS. 1 to 3, the frame 12 can include a frame body 20, an internal frame component 22 connectable to the frame body 20 on a first side thereof (e.g., on an inner side) and a frame cover 24 adapted to be connected to the frame body 20 on a second side thereof (e.g., on an outer side). It is thus noted that, when wearing the goggles 10, the internal frame component 22 is adapted to face and/or engage with the user's face, such as around the eyes and across the bridge of the nose, for example, and the frame cover 24 is adapted to be coupled to the frame body, opposite the internal frame component 22. In some embodiments, parts of the frame cover 24 can extend through the frame body and/or be positioned on the first side, together with the internal frame component 22. In addition, a layer of foam material can be connected to and/or integrated with the internal frame component 22 to improve comfort, for example.

In some embodiments, the frame body 20 is made of a generally rigid material, such as plastic, to allow the frame body 20 to retain its overall shape. However, in other embodiments, the frame body 20 can be made of substantially flexible material. As noted, plastic can be used for its general rigidity while also allowing for some flexibility. As seen in FIG. 4, the frame body 20, the internal frame component 22 and the frame cover 24 include respective closed perimeters defining an opening 25 through which the user can see. As will be described further below, the lens assembly 14 is configured to be connected to the frame 12 to cover the opening 25 and protect the user's eyes, among others. It is appreciated that the shape of the frame body 20, the internal frame component 22 and the frame cover 24 are similar and/or at least partially complementarily shaped and configured to cooperate with one another to form a cohesive and well-structured frame 12.

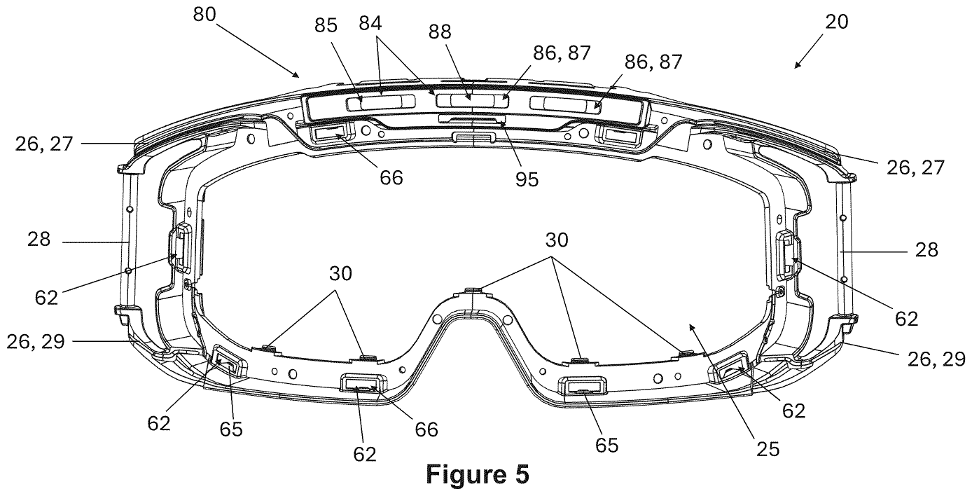

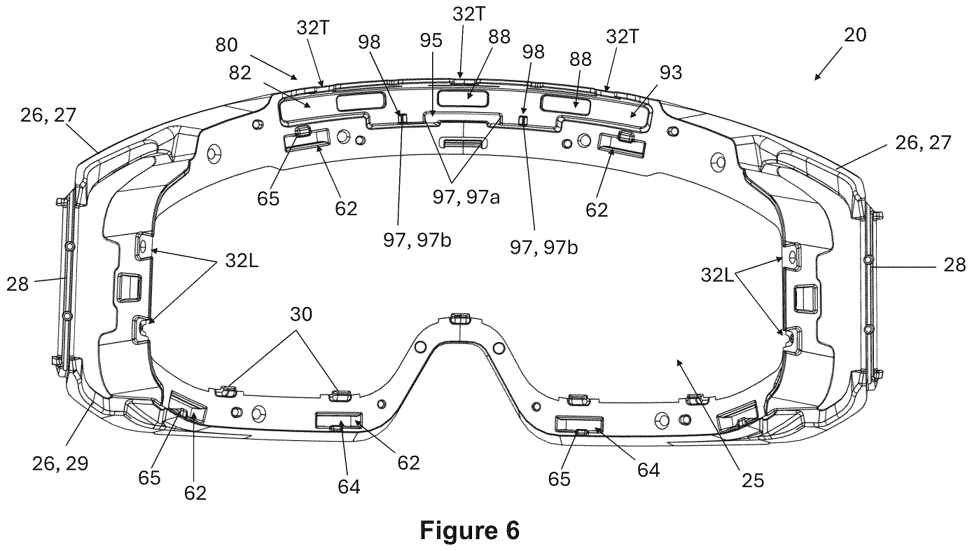

As seen in FIGS. 5 and 6, the frame body 20 further includes outriggers 26 extending therefrom proximate edges of the frame body 20. Outriggers typically include “arms” extending outwardly from the frame body 20. The outriggers 26 are designed to assist the goggles in mating with the user's face when wearing the helmet, which in turn provides a more comfortable and secure fit. More specifically, the outriggers are configured to enable adjusting the goggles relative to the user's face instead of relative to the helmet shell. In other words, without the outriggers, operating the straps to adjust the fit of the goggles would result in a force being applied against the sides of the helmet shell (e.g., instead of against the user's face). In other words, the outriggers 26 extend the strap attachment points outward, allowing the goggles to sit deeper into the helmet and creating a better seal against the face of the user.

Known goggles have flexible (e.g., bendable and/or twistable) frame bodies, which can at least partially conform to the user's face and prevent at least some damage to the frame when subjected to forces in flexion, torsion or otherwise. However, such forces can cause the lens to “pop out”, for example, during high-impact activities, or when riding over rough terrain. Moreover, when attempting to remove the lens from a flexible/twistable frame, the seal created by the goggles within the front opening of the helmet shell can be inadvertently broken, which can be tiresome, and even troublesome at times. These known goggles often include locking mechanisms to secure the lens to the frame and prevent accidental disconnections.

The improved seal (e.g., provided by the outriggers 26) and rigid configuration of the frame body 20 of the present disclosure enable disconnection of the lens assembly 14 therefrom without (or with minimal) deformation of the frame body 20. The seal with the helmet and/or against the user's face is maintained, and the lens assembly 14 can be reconnected to the frame body 20 when desired using the corresponding attachment system, which will be described further below. In addition, the foam layer of the internal frame component 22 can provide flexibility to the goggles to enable at least partially conforming to the user's face. In other words, the foam layer can improve ergonomics of the goggles by enabling the goggles to conform to different users having respective head shapes and sizes.

With continued reference to FIGS. 5 and 6, in some embodiments, the outriggers 26 can also increase comfort of the user by enabling connecting the straps further away from the face of the user, which can reduce pressure points. Moreover, having the outriggers extend away from the frame can also increase peripheral vision of the user as the straps and/or frame body no longer overlap the lens (e.g., on lateral sides thereof). The design of the outriggers 26 can also improve airflow around the frame body 20 (e.g., around the goggles), which assists in reducing accumulation of snow, frost, fog, moisture, etc.

In this embodiment, the frame body 20 includes top outriggers 27 extending from opposite ends of a top edge of the frame body, and bottom outriggers 29 extending from opposite ends of a bottom edge of the frame body 20. Each pair of top and bottom outriggers are connected to one another via respective outer connectors 28, thereby defining a closed outer perimeter of the frame body 20. The outriggers 26 are integrally formed as part of the frame body 20 such that the frame body and outriggers form a single, one-piece unit. However, it is appreciated that other configurations are possible, such as having the outriggers be a separate component and removably connectable to the frame body, for example. In some embodiments, the strap clips 18 are configured to be removably connected to respective outer connectors 28, although other configurations are possible.

In some embodiments, the frame body 20 can include a ventilation system 80 configured to improve airflow around, through and/or within the goggles. It is appreciated that improved airflow can assist in preventing obstruction of the lens, for example, due to accumulation of fog, frost/ice, moisture, snow, etc. As seen in FIGS. 4 to 6, the ventilation system 80 of the frame body 20 can include a frontal ventilation 82 provided across a top edge of the frame body 20. The frontal ventilation 82 includes one or more openings 84 defined through the structure of the frame body 20 such that air is allowed to flow through the openings 84. In other words, the air is allowed to circulate, among others, via the openings 84, between an environment surrounding the goggles (e.g., outside/on a first side of the lens) and within the goggles (e.g., inside/on a second side of the lens/between the lens and the user's face).

In this embodiment, the frontal ventilation 82 includes three openings 84 spaced along the top edge of the frame body 20. The openings can be distributed evenly (e.g., at regular intervals) across the top edge or following any other suitable configuration. In some embodiments, the openings 84 have a generally tapered shape, with an area of the opening reducing from a first end (e.g., proximate the outer side) to a second end (e.g., proximate the inner side) of the opening. This configuration of the openings 84 defines a funnel 85 configured to drag air towards the opening 84 and improve airflow therethrough. The shape and size of the openings 84 are also adapted to compensate for a loss of airflow caused by a filter component, which will be discussed further below. As such, by enlarging the wider part of the funnel 85, the filter surface area can be increased in order to enable admission of an airflow as close as possible to a maximum airflow allowed by the size of the narrower part of the funnel 85. The reduction of the area (i.e., the taper) of the opening 84 can be gradual, for example, using slanted walls tapering inwardly. Alternatively, and as seen in FIGS. 4 and 5, the opening 84 can have a stepped or segmented reduction of area.

In this embodiment, the opening 84 includes an inset region 86 defined in a thickness of the frame body 20, and a hole 88 defined within the confines of the inset region 86. Specifically, the inset region 86 includes an oblong depression 87 defined along the top edge of the frame body. The oblong depression 87 has a first area. The hole 88 is defined through the frame body 20 and is located at least partially within the first area (e.g., within the oblong depression). As such, it is understood that the hole 88 has a second area, which is smaller than the first area. Therefore, the reduction of the area of the opening 84 is defined by the sequential first and second areas. In this embodiment, the hole 88 is shorter than the oblong depression 87 and is generally as wide (i.e., the reduction in size is along a single direction). However, it is appreciated that other configurations are possible and may be implemented for the frontal ventilation.

Referring back to FIG. 4, the frontal ventilation 82 can include a filter component 90 adapted to overlay the one or more openings 84. The filter component 90 is configured to allow air to flow therethrough while preventing, or at least decreasing the amount of water, dirt, snow and debris from the air passing through (e.g., flowing through the openings 84). In some embodiments, the filter component 90 corresponds to a Frogzskin® mesh liner, although other configurations are possible, such as a nylon mesh, for example. The filter component 90 can be a monolithic liner shaped and sized to cover every opening of the frontal ventilation 82. Alternatively, the filter component 90 can include a plurality of liners positioned to cover respective openings 84. In this embodiment, the filter component 90 is installed on the outer side of the frame body 20. As will be described below, the frame cover 24 can be adapted to cover and protect the filter component 90 upon connection thereof with the frame body 20. For instance, the connection of the frame cover 24 can ensure that the filter component 90 remains in place (e.g., overlaid on the openings 84).

In some embodiments, the frontal ventilation 82 is operable between an open configuration, where air is allowed to flow through the openings 84, and a closed configuration, where air is prevented or obstructed from flowing through the openings 84. In this embodiment, the frontal ventilation 82 includes a vent actuator 92 operable to change the frontal ventilation between the open and closed configurations. The vent actuator 92 is manually operable such that the openings 84 can be opened and closed when desired. With reference to FIG. 6, the frame body 20 includes a frontal recess 93 defined along the top edge on the inner side (e.g., opposite the filter component 90). In this embodiment, the holes 88 of each opening 84 open within the frontal recess 93. However, other configurations are possible, such as providing the frame body 20 with openings next to the frontal recess, for example.

As seen in FIGS. 4 to 6, the vent actuator 92 includes a slider 94 shaped and sized to engage the frontal recess 93. The slider 94 is slidably coupled to the frame body 20 within the frontal recess 93 and is operable to selectively cover and uncover the openings 84 (i.e., to obstruct and allow airflow through the openings). In this embodiment, the slider 94 is adapted to be slid from side to side along the frontal recess to correspondingly open and close the frontal ventilation. Specifically, the slider 94 can be provided with slider openings 94a configured to be selectively aligned with the openings 84 of the frontal ventilation, thereby enabling airflow therethrough.

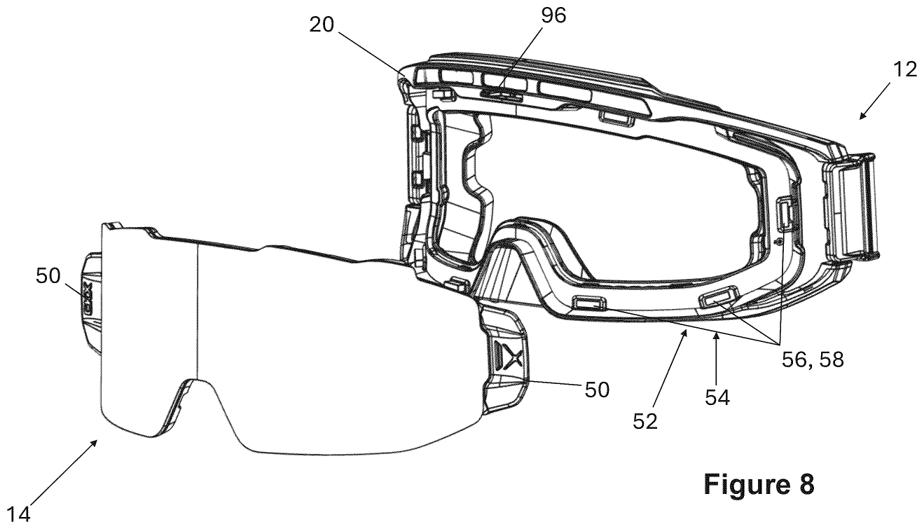

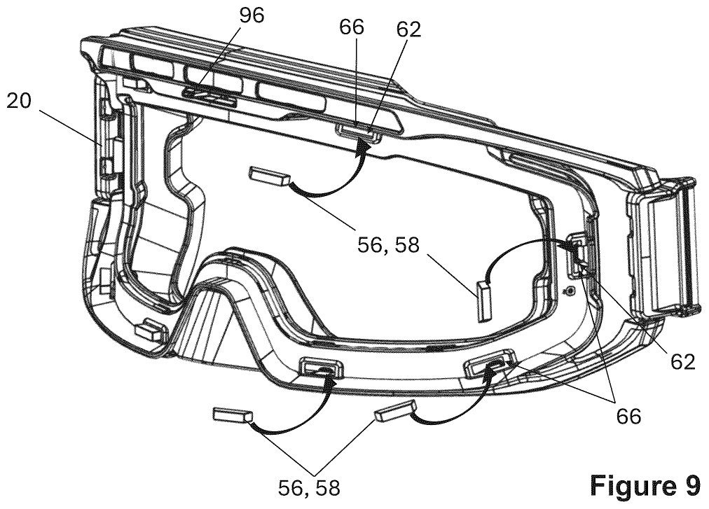

Upon sliding the slider 94, the structure of the slider is configured to obstruct and/or completely occlude the openings 84 to block airflow therethrough. The slider 94 can include a slider actuator 96 extending therefrom and configured to be manipulated to facilitate operation of the slider 94. Specifically, the frame body 20 can include an actuator slot 95 defined through the frame body and opening from the outer side and into the frontal recess 93, such as below the openings 84. The slider actuator 96 can be adapted to extend through the actuator slot 95 to enable operation thereof from an exterior of the goggles (e.g., from the outer side of the frame body). This configuration is shown in FIGS. 8 and 9, among others. It is thus noted that the user can manually operate the slider, for example, with a finger, by pushing against the slider actuator 96 to engage the slider 94 in movement along the frontal recess 93 to change the configuration of the frontal ventilation 82.

The frontal recess 93 and/or the slider 94 can be provided with structural components defining predetermined locations for operational positions of the slider 94. For instance, at least one of the frontal recess 93 and the slider 94 can include one or more detents 97 configured to hold the slider 94 in respective predetermined locations. The detents 97 can assist in holding the slider in the predetermined locations until sufficient force is applied to disengage the detents and allow movement of the slider. In some embodiments, the vent actuator 92 can include one or more detents 97 configured to assist in positioning the slider 94 in fully-open and fully-closed positions. The vent actuator 92 can include additional detents 97 to allow positioning the slider 94 in one or more intermediate positions, such as partially closed (e.g., >50% closed), partially open (e.g., >50% open) and/or evenly open/closed (e.g., 50% open/50% closed). As used herein, it should be noted that the expression “detent” can refer to a device (or feature of a device) configured to assist in positioning mechanical parts relative to one another. Exemplary embodiments of detents include, but are not limited to, catches (e.g., recesses, slots, nooks, corners, etc.), dogs and springs, among others.

In this embodiment, the detents 97 include central detents 97a positioned at opposite ends of the actuator slot 95. More specifically, the central detents 97a and the slider actuator 96 can cooperate to define two predetermined positions of the slider 94, such as the fully-open and fully-closed positions. More particularly, in this embodiment, positioning the slider actuator 96 in abutment with a first one of the central detents 97a configures the slider 94 in the fully-open position, thereby fully opening the frontal ventilation 82. Similarly, positioning the slider actuator 96 in abutment with a second one of the central detents 97a configures the slider 94 in the fully-closed position, thereby fully closing the frontal ventilation 82. In some embodiments, the slider 94 can be adapted to abut against sidewalls of the frontal recess 93 when in the fully-open and fully-closed positions. The frontal recess 93 is therefore shaped and sized to define additional stops for the slider 94 to further assist in positioning the slider 94 in the desired predetermined location.

In some embodiments, the slider 94 and the frame body 20 can include additional components configured for guiding the movement of the slider 94 and/or maintaining the slider 94 coupled to the frame body (e.g., within the frontal recess). For instance, and with reference to FIGS. 4 and 4B, the slider 94 can include one or more hooks 101 configured to engage respective slots 105 defined in the frame body 20. The slots 105 are elongated to enable the hooks 101 to slide therealong during movement of the slider 94 along the frontal recess 93. The shape of the hooks 101 also assists in maintaining the slider 94 connected to the frame body 20. The hooks 101 can be resilient elements adapted to be pushed down (e.g., manually) through respective slots 105 in order to enable disconnection of the slider 94 from the frame body 20. It is thus noted that the slider 94 can be slidably, removably and operably connected to the frame body 20.

The detents 97 can also include one or more lateral detents 97b configured to define at least one intermediate predetermined position. As seen in FIGS. 4B and 6, the frontal recess 93 can be provided with one or more indentations 98, and the slider 94 can include one or more protuberances 99 shaped and sized to engage with respective indentations 98 in order to retain the slider 94 in the desired position (e.g., in the intermediate predetermined position). It is noted that, in another embodiment, the indentations 98 can be provided on the slider 94, and that the protuberances 99 can correspondingly be provided on along the frontal recess 93. In yet another alternative embodiment, each one of the frontal recess and the slider can be provided with one of each lateral detent component. In other words, each one of the frontal recess 93 and the slider 94 can include one indentation 98 and one protuberance 99. In this embodiment, and as seen in FIG. 6, the lateral detents 97b are provided on each side of the actuator slot 95. It is thus noted that, when in the intermediate predetermined position, the slider actuator 96 is positioned between (e.g., halfway) between the central detents 97a, such as partway across or in the middle of the actuator slot 95. From the above, it is noted that, in this embodiment, the slider 94 is operable in three (3) predetermined positions, defined (or assisted) by the detents 97.

In some embodiments, and as seen in FIG. 4B, the vent actuator 92 (e.g., the slider 94) can have a monolithic structure. It should be noted that, as used herein, the expression “monolithic structure”, or alternatives thereto, can refer to a component which does not include interconnected and/or disconnectable parts forming a whole, but rather a one-piece unit. It should also be noted that, in this embodiment, while the monolithic structure can have different portions, sections and/or features, these different portions cannot be disconnected from one another as they are part of the same monolithic structure. However, it is appreciated that other configurations are possible, such as using several (e.g., two or more) pieces connected together to define a modular and/or adjustable slider. In this embodiment, the monolithic structure includes, among others, the body of the slider 94, the protuberances 99, the slider actuator 96 and the hooks 101.

In this embodiment, the internal frame component 22 is adapted to at least partially cover the inner side surface of the frame body 20. The internal frame component 22 can be made of a softer material, such as rubber or foam, to improve comfort of the user wearing the goggles, for instance. The internal frame component 22 is removably connectable to the frame body 20 to enable disconnection for removal, maintenance and/or replacement thereof. For instance, different embodiments and/or configurations of the internal frame component 22 can be provided, such as internal frame components of varying materials, a ventilated internal frame component, an isolated internal frame component, an internal frame component with a wider or narrower nose bridge, etc. It is therefore noted that the desired internal frame component can be chosen and connected to the frame body 20, then subsequently removed and replaced, as needed and/or desired.

In some embodiments, the frame body 20 can be provided with integrated ventilation features (e.g., the fontal ventilation 82) such that the internal frame component 22 can include or omit ventilation features. For example, the internal frame component 22 can include redundant and/or complementary ventilation features to those of the frame body 20. Alternatively, the internal frame component 22 can be “non-ventilated”, for example, if the frame body 20 provides sufficient ventilation. The internal frame component 22 can be adapted to cover and at least partially seal interstices, openings and/or slots defined in the frame body 20. For example, the internal frame component 22 can have a top part shaped and sized to cover the slots 105, among other openings, as will be described further below.

In some embodiments, the internal frame component 22 can include some electronic elements (e.g., components of the heating assembly). As will be described further below, the heating assembly, or at least components thereof, can be coupled and/or integrated to the frame body 20 such that the internal frame component 22 can be removed and/or replaced without affecting the heating assembly. The internal frame component 22 can be connected to the frame body 20 using any suitable method, such as via fasteners (e.g., screws, bolts, etc.) or via clips. As seen in FIGS. 4 to 6, the frame body 20 can include a plurality of clip heads 30 extending from the inner side surface, and the internal frame component 22 can include clip openings 32 defined therethrough. The clip heads 30 are configured to engage respective clip openings 32 to establish and maintain the connection between the frame body 20 and the internal frame component 22. It is appreciated that other configurations and/or components are possible and may be used, such as having clip heads extending from the internal frame component 22 and clip openings defined on the frame body, for example. Additional and/or complementary clips and corresponding clip components can be provided along any other suitable sections of the frame body and the internal frame component to facilitate and/or improve the connection therebetween.

In this embodiment, the frame body 20 has lateral clip openings 32L and top clip openings 32T defined along respective lateral and top edges thereof. The internal frame component 22 correspondingly has lateral and top clip heads 30L, 30T configured to engage with the clip openings of the frame body. The lateral and top clip components assist in positioning the frame body 20 and the internal frame component 22 relative to each other while also assisting in establishing and maintaining sealed connections therebetween. As seen in FIG. 6, the frame body 20 has two lateral clip openings 32L on each side thereof, and three top clip openings 32T. As such, it is appreciated that the internal frame component is provided with a corresponding number of clip heads (e.g., two lateral and three top clip heads), although other configurations are possible. It should be understood that the different clip components are designed to facilitate manufacturing (e.g., via 3D printing, moulding, etc.) and assembly by allowing the different parts of the frame 12 to be manually pressed and clipped together to establish the required connections.

In this embodiment, the frame cover 24 is shaped and sized to generally match at least a portion of the frame body 20 to facilitate connection therebetween. As will be described further below, the frame body 20 is shaped and configured to house and/or retain various components, such as electrical components of the heating system, and the frame cover 24 is configured to cover and protect these components. As seen in FIG. 4, the frame cover 24 includes outrigger covers 34 configured to cover respective outriggers 26 of the frame body 20. In this embodiment, the frame cover 24 corresponds to an overmolded part of the frame body 20 such that the frame cover 24 becomes an integrated part of the frame body 20. In other words, the frame cover 24 is molded on top of the frame body 20, thereby becoming inseparable therefrom and sealing the electrical components between the frame body 20 and the frame cover 24. However, it should be noted that other configurations are possible, such as having the frame cover 24 be removably connected to the frame body 20 to enable selective access to the electrical components housed therein. In some embodiments, the frame cover 24 defines an interface between the frame 12 and the lens assembly 14. As such, the frame cover 24 can be made of a generally soft material adapted to enable sealing interstices between the frame 12 and the lens assembly 14. It is appreciated that the frame cover 24 can correspond to a gasket on which the lens assembly 14 sits when connected to the frame 12. As noted above, the frame cover 24 can be an integrated part of the frame body 20 such that the gasket corresponds to an integrated gasket.

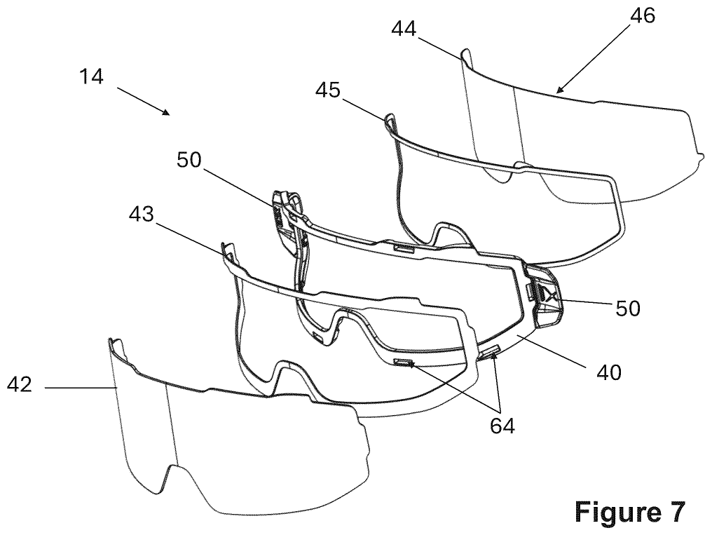

With reference to FIG. 7, the lens assembly 14 illustratively includes a lens frame 40, an external lens 42 and an internal lens 44. The external and internal lenses 42, 44 are connectable to the lens frame 40, which is in turn connectable to the frame 12 (e.g., to the frame body 20). In this embodiment, the external lens 42 is connectable to the lens frame 40 via an external lens adhesive layer 43 (e.g., double-sided adhesive). Therefore, the external lens adhesive layer 43 is affixed to a first side (e.g., an external side) of the lens frame 40, and the external lens 42 can then be connected to the external lens adhesive layer 43 to mount the external lens 42 to the lens frame 40. Similarly, the internal lens 44 is connectable to the lens frame 40 via an internal lens adhesive layer 45.

The external lens 42 can correspond to a protective component of the lens assembly, such as a lens made of polycarbonate adapted to prevent damages, including scratches and/or scrapes, for example. In some embodiments, the external lens 42 can be coated or laminated to correspond to an “anti-scratch” lens. The external lens 42 can further be adapted to protect the user from impacts, debris and projections, and against the sun's UV light, among others. In this embodiment, the internal lens 44 is laminated/coated to prevent fog accumulation thereon. The internal lens 44 includes at least one heating surface 46 (e.g., an outer surface, an inner surface, an intermediate surface, or any combination thereof) operatively connectable to the heating system of the goggles 10 for providing heat to the lens assembly 14 (e.g., via the internal lens 44). The intermediate surface can correspond to positioning the heating surface 46 between layers of PET (Polyethylene Terephthalate). It is noted that the internal lens 44 can therefore correspond to a conductive lens of the lens assembly 14. It should also be noted that the expression “conductive lens” can refer to an ability of the internal lens to conduct an electric current across a surface thereof via conductive elements in a manner that will be described below.

In some embodiments, the lens frame 40 includes at least one lateral tab 50 extending from a lateral side thereof. The lateral tab 50 is configured to facilitate manipulation of the lens assembly 14 by providing a readily accessible part of the lens assembly 14 spaced from the lenses. As such, the external and internal lenses are protected from having fingerprints and smudges thereon from having to manipulate them directly, for instance. In some embodiments, the lateral tabs 50 can be oversized, e.g., larger than parts of the frame body 20 and/or one of the largest components of the lens assembly 14, to facilitate grasping the lateral tabs and manipulating the lens assembly 14. For example, the oversized lateral tabs 50 can facilitate manipulation thereof when the user is wearing gloves. As seen in FIG. 7, the lateral tabs 50 can be overmolded on the lens frame 40 and can extend along generally an entire side of the lens frame 40, although other configurations are possible, such as connecting the lateral tabs via mechanical fasteners (e.g., screws, etc.), adhesive (e.g., glue) and/or stitches. In this embodiment, the lens frame 40 includes a pair of lateral tabs 50 extending from opposite lateral sides of the lens frame. In some embodiments, the lateral tabs 50 can be shaped and sized to enable air deflection away from the goggles 10, and can therefore prevent air flow into undesired locations, such as into the helmet (e.g., through interstices of the goggles and/or between the goggles and the helmet).

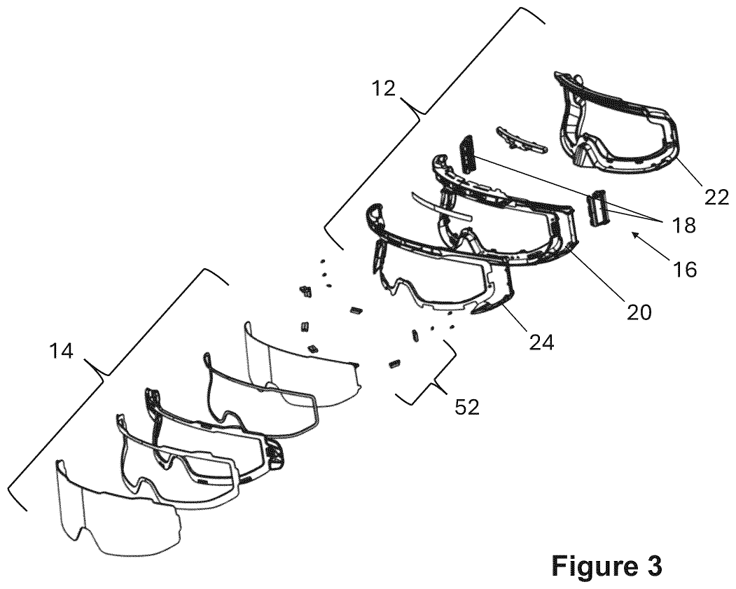

With reference to FIGS. 8 to 10, and with continued reference to FIGS. 3 to 6, the goggles 10 include an attachment system 52 configured to enable selective connection of the lens assembly 14 to the frame 12. More specifically, the lens assembly 14 is removably connectable to the frame 12 via the attachment system 52, and can be manipulated (e.g., connected and disconnected) using the lateral tabs 50, for example. In this embodiment, the attachment system 52 includes a magnetic connection assembly 54 coupled to one or both of the frame 12 and the lens assembly 14 for enabling magnetic connection of the frame 12 and the lens assembly 14 (e.g., attraction by opposite poles and/or attraction by a pole and a ferromagnetic material). The magnetic connection assembly 54 can include one or more magnets 56 coupled to the frame 12 and/or the lens assembly 14. It is noted that magnets 56 can be provided on the frame 12 to enable metallic elements of the lens assembly 14 to magnetically connect with the frame 12, and/or vice versa.

In this embodiment, the magnetic connection assembly 54 includes frame magnets 58 and lens magnets 60 configured to be connected to the frame 12 and the lens assembly 14, respectively. The frame magnets 58 are configured to engage respective lens magnets 60 to enable connection therebetween. In other words, in this embodiment, the frame 12 and the lens assembly 14 include the same number of magnets 56 such that the magnetic connection is established between pairs of frame and lens magnets 58, 60.

As seen in FIGS. 5, 6 and 8, the frame body 20 can be provided with magnet slots 62 shaped and sized to house respective frame magnets 58. More specifically, in this embodiment, the magnet slots 62 extend through a thickness of the frame body 20. The magnet slot therefore includes a slot inlet 64 defined on a first side of the frame body, such as on the inner side, and a slot outlet 66 defined on a second side of the frame body, such as on the outer side. The frame magnet 58 is adapted to engage the magnet slot 62 via the slot inlet 64 to have a portion thereof proximate the slot outlet 66 to enable cooperation with a corresponding one of the lens magnets.

In some embodiments, the internal frame component 22 can be coupled to the frame body 20 to secure the frame magnets 58 within respective magnet slots 62, although other configurations are possible. In this embodiment, each magnet slot 62 can be provided with a slot clip 65 extending at least partially in front of the slot inlet 64 and configured to retain the magnet within its slot. In some embodiments, the frame cover 24 can be shaped and sized to avoid going over the slot outlets 66 on the outer side of the frame body 20. This configuration at least partially prevents affecting (e.g., reducing) the magnetic connection between the frame and the lens assembly, although other configurations are possible, such as using the frame cover 24 to cover the magnet slots to ensure the magnets are secured within respective slots. As previously noted, the frame cover 24 can be overmolded onto the frame body such that the magnet slots (e.g., the slot outlets 66) can be covered during the manufacturing process. However, and as seen in FIG. 4, the frame cover 24 is shaped and sized to avoid occlusion of the magnet slots.

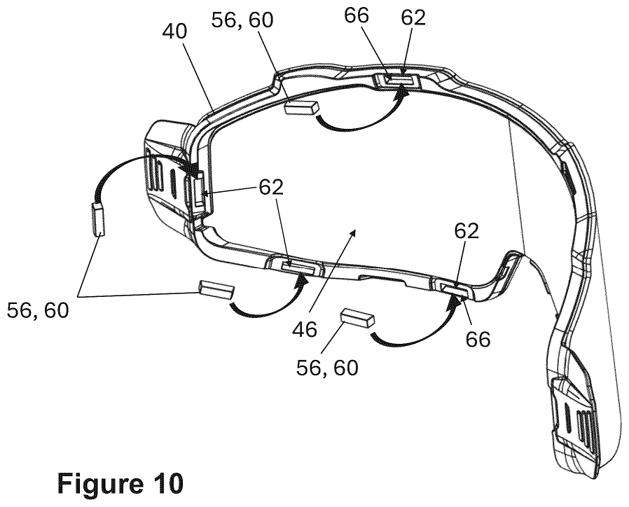

As seen in FIGS. 7 and 10, the lens frame 40 can be similarly provided with magnet slots 62 shaped and sized to house respective lens magnets 60. The magnet slots 62 can extend through a thickness of the lens frame 40. Each magnet slot therefore has a slot inlet 64 defined on a first side of the lens frame 40, such as on the outer side (e.g., on the side of the external lens 42), and a slot outlet 66 defined on a second side of the lens frame 40, such as on the inner side (e.g., on the side of the internal lens 44). The lens magnets 58 are adapted to engage respective magnet slots 62 via the slot inlets 64 to have a portion thereof proximate the slot outlets 66 to enable cooperation with a corresponding one of the frame magnets. In some embodiments, the external lens adhesive layer 43 can be coupled to the lens frame 40 in order to secure the lens magnets 60 within respective magnet slots 62, although other configurations are possible. In some embodiments, the internal lens adhesive layer 45 can be shaped and sized to avoid going over the slot outlets 66 on the inner side of the lens frame 40. This configuration can at least partially prevent affecting the magnetic connection between the lens assembly and the frame, although other configurations are possible.

In this embodiment, the frame body 20 illustratively includes eight (8) magnet slots 62, including four (4) magnet slots disposed along the bottom edge (e.g., two magnet slots on each side of the nose bridge), two (2) magnet slots disposed along the top edge, and one (1) magnet slot on each lateral side. It should therefore be readily understood that, in this embodiment, the lens frame 40 also includes eight (8) magnet slots 62 at similar locations as those of the frame body 20 to enable magnetic connection. However, it is appreciated that this configuration is exemplary only and that other configurations are possible, such as providing a different number of magnet slots disposed at any other suitable location, for example.

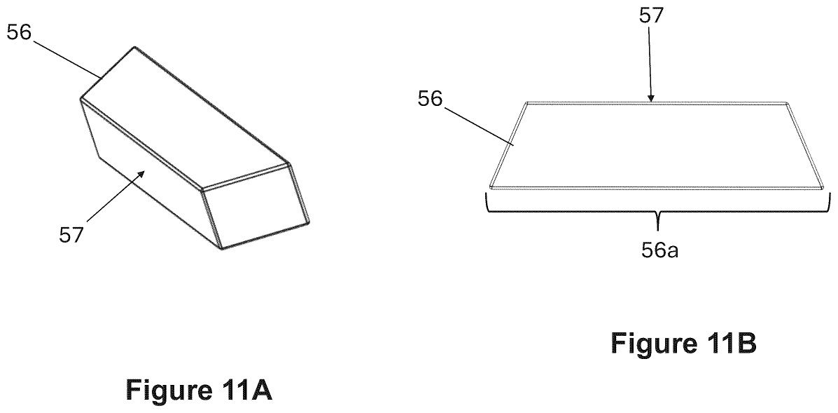

In this embodiment, the magnet slots 62 and magnets 56 can be at least partially complementarily shaped to facilitate cooperation therebetween. For instance, and as seen in FIGS. 11A and 11B, the magnets 56 can have a generally trapezoidal shape or a truncated pyramidal shape having a wider base region 56a, for example. The magnet slots 62 can have complementing shapes with regards to the trapezoidal magnets such that the wider base region prevents the magnets 56 from extending completely through the frame body 20 and/or lens frame 40. Moreover, the complementing shapes of the magnets 56 and magnet slots 62 improves cooperation between these components by allowing the magnets to “sit” in respective magnet slots. The magnet 56 also includes a forward surface, or connection surface 57, adapted to be positioned proximate the slot outlet 66 when the magnet is positioned within a magnet slot. It is thus noted that the connection surfaces 57 of each pair of frame and lens magnets 58, 60 are configured to engage one another to magnetically connect the lens assembly 14 to the frame 12.

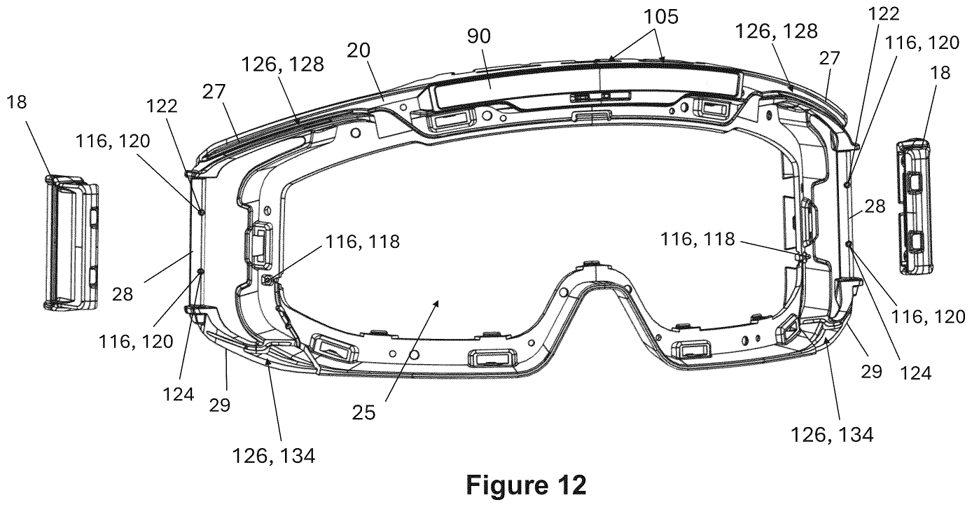

With reference to FIGS. 12 to 15, the heating assembly 100 is operable to provide energy, for example, in the form of an electric current, to the lens assembly 14 for increasing the temperature of the internal lens 44, or at least of the heating surface 46. In this embodiment, the heating assembly 100 is an electric heating assembly and is therefore configured to be operatively coupled to an electric power source. The power source can be small and/or transportable, such as a battery adapted to be carried by the user, for example, in a pocket, in a backpack, clipped on apparel, such as a jacket, a hat, a helmet or coupled to the strap of the goggles. In other embodiments, the power source can correspond to a fixed power source, such as an electric outlet of a vehicle, such as a snowmobile, for instance. In some embodiments, a receptacle can be coupled to the helmet shell (e.g., adhered, fastened or integrally formed with) and adapted to have the power source be connected (e.g., clipped) thereto.

In this embodiment, the goggles are battery-powered and configured to function with 7.4V and 12V batteries. In other words, the structure and configuration of the goggles (e.g., of the heating assembly 100) can operate properly (e.g., help prevent fog accumulation) using lower voltages (e.g., 7.4V) and can also operate and withstand higher voltages (e.g., 12V). In some embodiments, the resistance across the lens is such that the heating assembly 100 is configured to operate and sustain a 12V voltage without overheating. As such, it should be understood that the lens can also be heated when subjected to a voltage of 7.4V, although the corresponding heating process can be slower/longer. However, the relatively even heat distribution provided across the lens by the heating assembly 100 allows for such lower voltages (e.g., 7.4V) to still prevent fog accumulation thereon.

The heating assembly 100 can include a resistive structure, typically a metallic resistive structure, and conductive elements 102 provided on or across the heating surface 46. The conductive elements 102 can act as positive and negative terminals between which an electrical current can travel. For example, the conductive elements 102 can include bus bars 103 and/or electrodes 104 provided at opposite lateral ends 70 of the heating surface 46. As such, the electrical current can be provided to a first electrode 104, travel across the heating surface 46 via the resistive structure and reach a second electrode 104 in order to provide heat to the heating surface, and therefore to the lens. It should be noted that the electrodes 104 are laterally-positioned such that the electrical current travelling therebetween correspondingly travels across the lens (e.g., from left to right and/or from right to left).

In some embodiments, the bus bars 103 and/or electrodes 104 can be printed on the heating surface 46 using conductive ink, which typically includes paint containing silver and/or carbon particles. Conductive inks, and associated technologies, are known, such as in U.S. Pat. Nos. 9,777,167 and 9,777,168, and International PCT application No. PCT/US2021/054845, which are incorporated herein by reference in their entirety. It is thus noted that the electrodes 104 can be an integrated part of the internal lens 44. Alternatively, or additionally, the electrodes 104 can include rivets to define contact points to facilitate establishing the electrical connection. It is noted that using rivets can help avoid damaging the conductive ink, which can be delicate and be worn down over time and repeated use. Similarly, the resistive structure can include a conductive film, such as a transparent conductive film 160 (TCF), applied to a surface of the internal lens 44 (e.g., to the heating surface 46). The TCF can include a base, or substrate, overcoated with a nanowire layer and a conductive layer, the conductive layer including carbon nanotubes, copper micro mesh, silver nanowires and a binder. The TCF can therefore be adapted to provide electrical conductivity and high visible light transmission (e.g., high visibility). TCF and associated technologies are known in the art, such as in U.S. Pat. Nos. 11,393,607 and 11,933,865, and International PCT application No. PCT/US2021/054845, which are incorporated herein by reference in their entirety.

It is noted that a length of the lens (e.g., a distance between lateral ends 70) is typically greater than a height (e.g., a distance between a top and bottom portions) thereof. As such, it should be understood that positioning the electrodes laterally (e.g., horizontally spaced) implies spacing the electrodes further from each other, for example, compared to electrodes positioned at the top and bottom of the lens (e.g., vertically spaced). In this embodiment, the configuration of the TCF is configured to improve conductivity of electrical current, thereby enabling the electrodes to be positioned laterally. The TCF is also configured to provide high visibility and enables heating the lens assembly with relatively low power input, such as between 2 W and 15 W power based on the power source (e.g., 7.4V and/or 12V). For instance, a 7.4V voltage can provide 4 W of power for heating the lens, while a 12V voltage can provide 12 W of power for heating the lens. In stark contrast, known heated lens have electrodes vertically spaced from each other since horizontally-spaced electrodes would require either some “denser” conductive elements across the lens, which would reduce visibility, and/or some greater electrical power input, which can cause heating issues (e.g., overheating) and power consumption issues. It should be noted that increasing the electrical power input can cause other related issues. For instance, using a power source of 20V can become a health hazard while also becoming a scarcity as such power sources are not common and/or readily available. From preliminary tests, it was determined that the goggles as described herein (i.e., provided with the heating assembly 100) consume between about 30% and 70% less energy and provides similar or increased amounts of heating to the lens as known heated lenses.

Now referring to FIGS. 12 to 15, the heating assembly 100 includes an electric system 110 configured to enable providing the electric current to the heating surface 46 (e.g., to at least one of the electrodes 104). In this embodiment, the electric system 110 includes a power source 112 (e.g., a battery 114), electrical wiring 115, connection pins 116 and/or printed electrical circuits. The electrical wiring 115 is configured to operatively connect the power source 112 to the connection pins 116 to provide electricity thereto. In this embodiment, the frame body 20 is configured to house at least some of the electrical wiring 115 and is provided with the connection pins 116. Therefore, upon connecting the lens assembly with the frame, the connection pins 116 are configured to engage with the electrodes 104. As such, an electrical connection is established, and the electrical current can travel across the heating surface 46 for heating up the internal lens. As will be described further below, the electric system 110, in cooperation with the heating surface 46, is configured to define a closed-loop electric circuit. In other words, connection of the lens assembly 14 with the frame defines the closed-loop electric circuit.

The electric system 110 can include internal connection pins 118 configured to engage and connect with the conductive elements 102 (e.g., the electrodes 104) of the internal lens. In this embodiment, the electric system 110 includes a pair of internal connection pins 118 provided on opposite sides of the opening 25 of the frame body 20. It is thus noted that the internal connection pins 118 can connect with respective electrodes 104 upon connecting the lens assembly with the frame. In some embodiments, the internal connection pins 118 can enable the connection of additional components, such as lights (e.g., LEDs), sensors, etc. The electric system 110 can also include external connection pins 120 configured to engage and connect with the power source 112 and/or other external components, such as sensors, lights, cameras, controllers, etc. In this embodiment, and as seen in FIG. 12, the external connection pins 120 extend from the outer connectors 28 (i.e., the structure extending between the top and bottom outriggers). More specifically, each outer connector 28 can be provided with a pair of external connection pins 120.

In some embodiments, each pair of external connection pins 120 can include an input connection pin 122 and an output connection pin 124 respectively connectable to positive and negative terminals, such as to a positive terminal 112a and a negative terminal 112b linked to the power source 112, to enable closing the electric circuit. In this embodiment, the strap clips 18 are configured to connect to the outer connectors 28 of the frame body 20 for connecting the strap 17 to the goggles. The strap clips can include electrical components configured to engage with the corresponding pair of external connection pins 120. For example, a first strap clip can be adapted to be connected to the power source such that the terminals 112a, 112b are operatively connected to the input and output connection pins 122, 124 upon connecting the strap clip 18 to the outer connector 28. In such embodiments, the strap clip 18 can include an electrical socket configured to enable connection with the power source 112. This configuration is described in Applicant's own U.S. Pat. No. 11,510,814, which is incorporated herein by reference in its entirety. It is noted that the electrical wiring 115 extending along the strap(s) 17 can be elastic and/or have elastic covering to prevent damaging the electrical wiring upon stretching the strap, for instance. Such embodiments, among others, are described in published applications, including US patent publication No. 2024/0082062, which is incorporated herein by reference in its entirety.

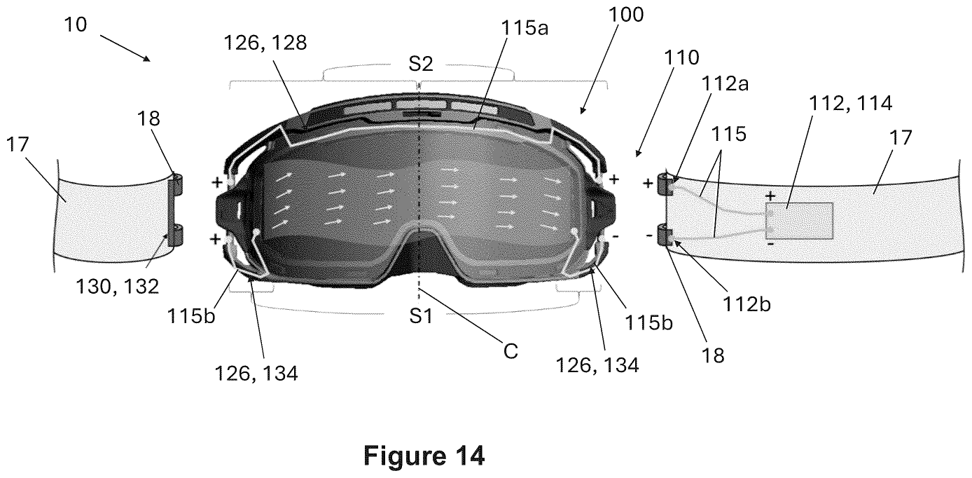

Referring more specifically to FIGS. 12 and 14, in this embodiment, the electrical wiring 115 can extend across the frame body 20 to enable connection between the internal connection pins 118 and the external connection pins 120. In this embodiment, the frame body 20 can include wire channels 126 shaped and sized to at least partially house the electrical wiring 115 therein. The wire channels 126 can include a top wire channel 128 extending across the top edge of the frame body to enable a top electrical wire 115a to establish an electric connection between opposite pairs of external connection pins 120. For instance, the top electrical wire 115a can be adapted to establish a connection between the pair of input connection pins 122. In this embodiment, the top wire channel 128 is defined along the top outriggers 27 such that the top electrical wire 115a can reach and engage each input connection pins 122 while remaining housed within the frame body 20 (e.g., within the wire channels 126).

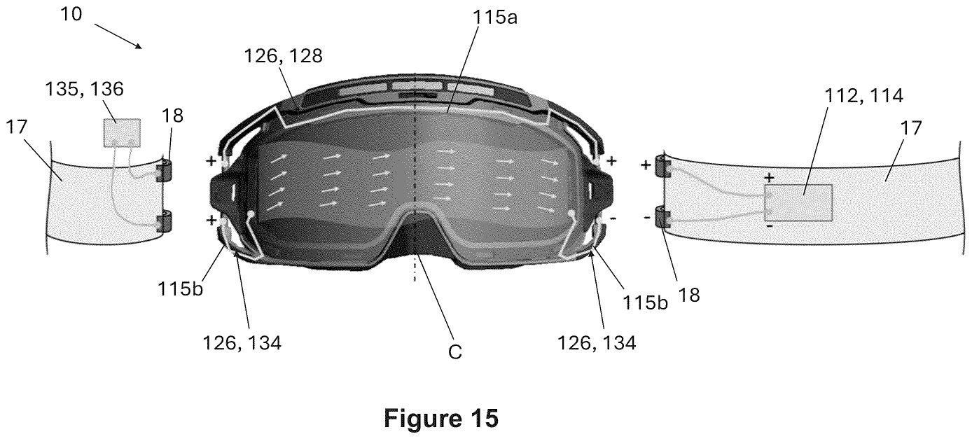

In this embodiment, the electric system 110 can include an electric connector 130 configured to establish an electric connection between the external connection pins 120 on a common side (e.g., between two external connection pins 120 coupled to a common outer connector 28). The electric connector 130 can include a jumper 132 configured to allow the electric current to “jump” from one connection pin to the other. It is thus noted that, in this embodiment, the external connection pins 120 on one side of the goggles are connectable to the power source 112, and the external connection pins 120 on the other side of the goggles are connected together. However, other configurations are possible. For example, and with reference to FIG. 15, the electric connector 130 can include a device 135, such as a controller 136 configured to be incorporated to the electric system 110, while maintaining the electric circuit closed and functional. It is thus noted that the device 135 can be powered by the same power source 112 configured to power the heating system 100. In this embodiment, the device 135 (e.g., the controller 136) is mounted directly on the strap 17 of the goggles, although other configurations are possible, such as mounting the device on the helmet or directly on the power source, for example. In some embodiments, the device 135 can alternatively, or additionally, include one or more lights (e.g., front light(s), back light(s), lateral light(s), etc.), a camera, a communication system (e.g., a microphone, speakers and an antenna, etc.), a projector, an ON/OFF switch, among other possibilities. It is appreciated that, while the device 135 is illustrated as being provided opposite the power source 112, other configurations are possible for incorporating the device to the electrical circuit. The device can, when desired and/or required, be connected in series or in parallel, based on the location of connection.

In some embodiments, the wire channels 126 further include bottom wire channels 134 extending between the outer connectors 28 and the lateral portion of the opening 25. As such, bottom electrical wires 115b can engage respective bottom wire channels 134 to establish a connection between the external connection pins 120 and the corresponding (e.g., nearest) internal connection pin 118. In this embodiment, the bottom wire channels 134 are defined along respective bottom outriggers 29 such that the bottom electrical wires 115b can extend between respective pairs of internal and external connection pins while remaining housed within the frame body 20 (e.g., within the wire channels 126). In some embodiments, each connection pin 116 corresponds to a pogo-pin which includes a spring-loaded pin head configured to facilitate establishing and maintaining the electrical communication between the connected components. For example, upon magnetically connecting the lens assembly to the frame, the electrodes of the internal lens are adapted to engage and “push” against the internal connection pins 118. The spring-loaded pin heads ensure that the electrical connection is maintained with the electrodes 104 as the magnets hold the lens assembly on the frame.

In this embodiment, and as illustrated in FIGS. 14 and 15, the closed electrical circuit can be defined as follows: the power source 112 provides the electrical current to the input connection pin 122, for instance, upon connecting the strap clip 18 to a first outer connector 28. The electric current then travels, via the top electrical wire 115a, to the input connection pin 122 of a second outer connector 28, and the jumper 132 or device 135 allows the electrical current to travel to the output connection pin 124 of the second outer connector 28. The electrical current then reaches the internal connection pin 118 on a first side of the opening 25 (e.g., proximate the second outer connector) via a bottom electrical wire 115b. The electrical current is then adapted to travel across the internal lens 44 when connecting the lens assembly 14 with the frame 12 (e.g., upon engaging the electrodes 104 with the internal connection pins 118). Finally, the electrical current travels from the internal connection pin 118 on a second side of the opening 25 (e.g., proximate the first outer connector) to the output connection pin 124 of the first outer connector via another bottom electrical wire 115b. The output connection pin 124 of the first outer connector being connected to the power source closes the electrical circuit.

Referring back to FIGS. 4 and 4A, the frame cover 24 is shaped and sized to cover the wire channels 126 in order to seal and protect the electrical wires therein. More specifically, the frame cover 24 includes the outrigger covers 34 which can be configured to cover at least some, or all of the wire channels 126 described above. It should thus be noted that the outrigger covers 34 can include top outrigger portions 140 configured to cover the top outriggers 27, including the top wire channels. Similarly, the outrigger covers 34 can include bottom outrigger portions 142 configured to cover the bottom outriggers 29, including the bottom wire channels. As described, the frame cover 24 can correspond to an overmold of the frame body 20 such that the electrical wires can be permanently sealed within the structure of the frame. It is appreciated that the various components of the goggles assist in reducing any apparent electrical wires by covering, sealing and/or integrating the wires within the structure of the goggles (e.g., within the frame 12). The risk of snagging electrical wires is greatly reduced or completely eliminated. The frame cover 24 also includes pin openings 144 defined along outer connector covers 145 and configured to enable the internal connection pins to extend through the frame cover 24. As such, electrical connection can be established with the lens assembly following the connection of the frame cover (e.g., after the overmolding process).

As seen in FIG. 14, the goggles 10 can have one or more axis of symmetry. For example, the lateral edges of the goggles, including the outriggers and the outer connectors 28, can be symmetrical relative to each other (e.g., relative to a centerline C). Alternatively, or additionally, substantially the entire structure of the goggles 10 can be symmetrical. The symmetry between these sections (S1, S2) enables certain components to be interchangeable and/or reversible, such as the bottom electrical wires 115b (interchangeable) and the top electrical wire 115a (reversible), for example. The symmetry also allows for the power source to be coupled to either side of the frame, therefore also enabling the electrical current to travel in both directions (e.g., across the internal lens from left to right and/or from right to left).

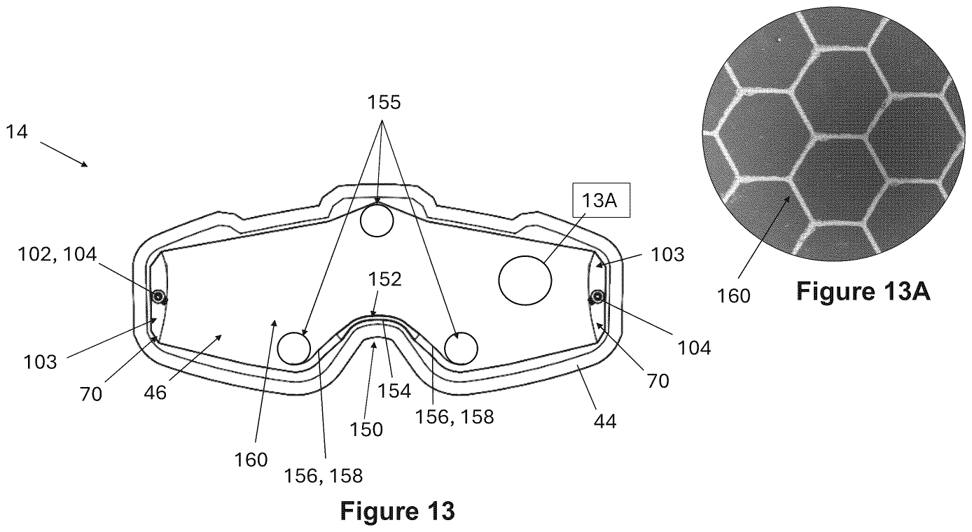

Now referring to FIG. 13, the heating assembly 100 is configured to provide heat to the lens assembly by generating an electrical current and having the electrical current travel across the internal lens between electrodes 104. It is thus noted that the electrodes 104 are laterally positioned and generally vertical (e.g., generally parallel with lateral sides of the frame) to enable the electrical current to travel across (e.g., between left and right sides) the internal lens. In other words, the electrical current can be adapted to travel in a general horizontal direction from one electrode to the other. It is also noted that the electrical current tends to favor a direct path of travel and will typically travel straight across the internal lens. In some embodiments, the direction of travel of the electric current, combined with the geometry of the internal lens, can create one or more cold spots. Typical cold spots are located proximate the nose bridge 150, such as on either sides thereof, which can correspond to the lowest regions of the lens, and thus to some of the furthest points from a center of the lens (through which the electrical current will favor travel). Similarly, another cold spot can be created proximate a top central area of the lens, which can correspond to the highest region of the lens.

In this embodiment, the internal lens 44 can be provided with an electrical conductor 152 installed proximate the nose bridge 150. The electrical conductor 152 is configured to bias the electrical current travelling across the internal lens towards the nose bridge 150 in order to heat up known cold spots 155 (e.g., typical cold spots on known designs). The electrical conductor 152 can include a metallic component 154 affixed to the heating surface of the internal lens and configured to have the electric current travel towards and/or along the metallic component 154. In some embodiments, the metallic component 154 can be printed onto the internal lens, such as via a screen-printing process using conductive inks (e.g., similar to the printing process of the electrodes).

Still referring to FIG. 13, the electrical conductor 152 can additionally or alternatively include a mechanical break 156 in the structure of the heating assembly 100, such as a break in the resistive structure (e.g., in the TCF). The mechanical break 156 is configured to bias the electrical current travelling proximate the break to go over and under the break. It is thus noted that creating the mechanical break above the nose bridge 150 can bias the electrical current to travel underneath the mechanical break 156 and proximate the typical cold spots, for instance. In this embodiment, the mechanical break can be defined by a kiss cut 158, where a cut is made across the internal lens 44 and through some layers thereof. It is appreciated that kiss cutting is a manufacturing technique that allows for cutting through one or more layers of the internal lens while leaving at least one layer intact. In other words, the created cut does not extend completely through the internal lens. In some embodiments, the kiss cut 158 disrupts the structure of the TCF 160 (e.g., locally breaks the electrical circuit), such as the nanowire layer and/or the conductive layer (e.g., the carbon nanotubes). The electrical current would therefore tend to travel across the internal lens along the undisrupted structure of the TCF 160, which includes portions below the kiss cut 158. One or more kiss cuts can be created at various locations on the internal lens 44 to bias the electrical current in desired portions thereof, such as known/typical cold spots, for example.

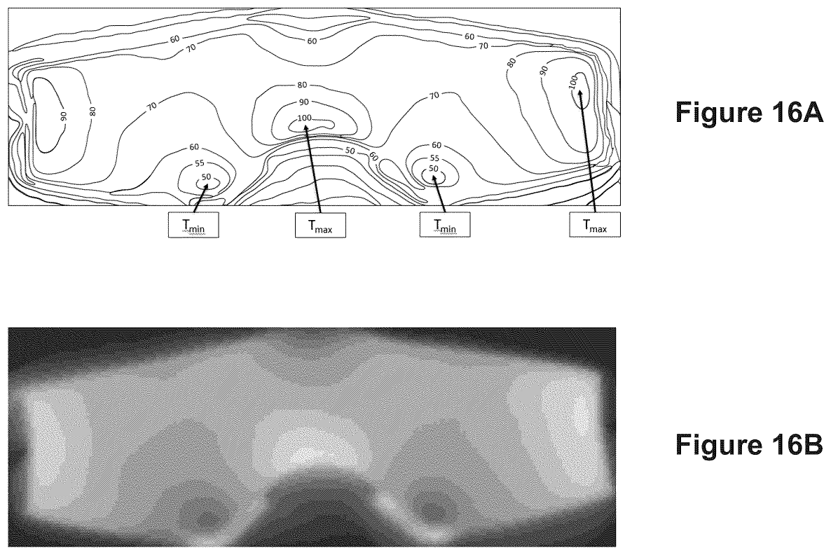

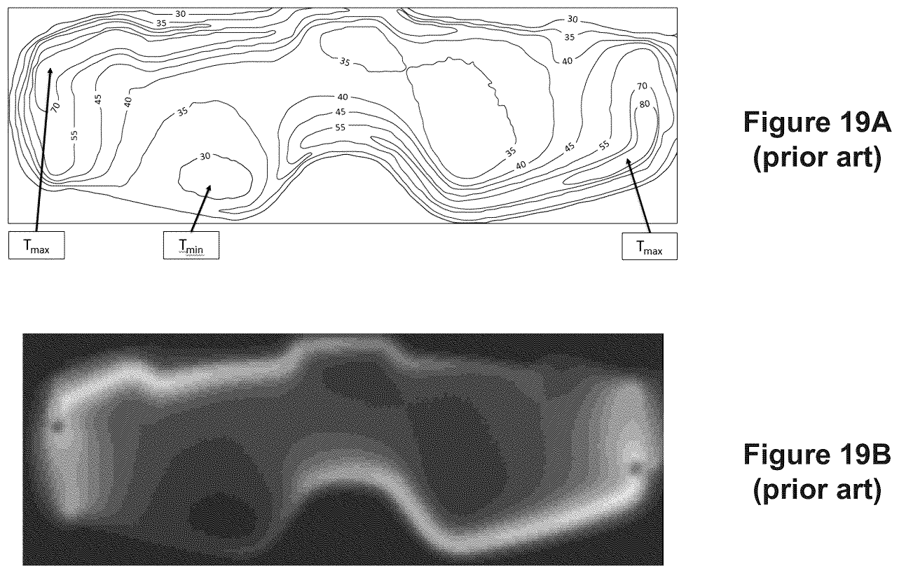

With reference to FIGS. 16A to 19B, various tests were performed to evaluate the performance of different embodiments of the electric goggles (i.e., provided with the TCF 160) against the performance of known electric goggles. As noted in Table 1 below, FIGS. 16A to 18B illustrate the temperature distribution across the lens of the electric goggles using the heating assembly disclosed herein with varying specifications (e.g., with different voltages (V), power (P), current (I), etc.). For comparison, FIGS. 19A and 19B illustrate the temperature distribution across the lens of a known pair of goggles.

TABLE 1
Performance evaluation and comparison

| | 16A and | 17A and | 18A and | 19A and 19B |
|---|---|---|---|---|
| Figures | 16B | 17B | 18B | (PRIOR ART) |
| V (V) | 12.0 | 7.5 | 5.0 | 12.0 |
| I (A) | 1.2 | 0.75 | 0.5 | 0.73 |
| P (W) | 14.4 | 5.6 | 2.5 | 8.8 |
| R (Ω) | 10.0 | 10.0 | 10.0 | 16.4 |
| Tᵣₒₒₘ (° C.) | 23.0 | 23.0 | 23.0 | 23.0 |
| Tₘₐₓ (° C.) | 102.3 | 63.7 | 47.9 | 103.2 |
| Tₘᵢₙ (° C.) | 47.7 | 36.8 | 31.2 | 27.7 |
| DT (° C.) | 54.6 | 26.9 | 16.7 | 75.5 |
| T_{avg} (° C.) | 71.0 | 49.7 | 39.3 | 45.4 |
| σ (° C.) | 12.8 | 6.7 | 4.3 | 14.5 |

As noted in Table 1 above, the embodiment shown in FIGS. 16A and 16B was tested using a 12V power source and a power input of 14.4 W, corresponding to about 164% of the power input of the prior art embodiment (shown in FIGS. 19A and 19B). It is noted that there are less temperature variations than in the prior art despite higher power. In addition, the average temperature is higher at 71.0° C. and the temperature distribution is more uniform over the surface of the lens (σ=12.8° C.). From FIGS. 16A, 16B and Table 1, we can also note that substantially the entire optical zone is well heated, at above 70° C., the center of the optical zone is between 70° C. and 80° C., the “cold spots” are in the expected locations (e.g., lower left/right corners near the nose), but are still at about 50° C.

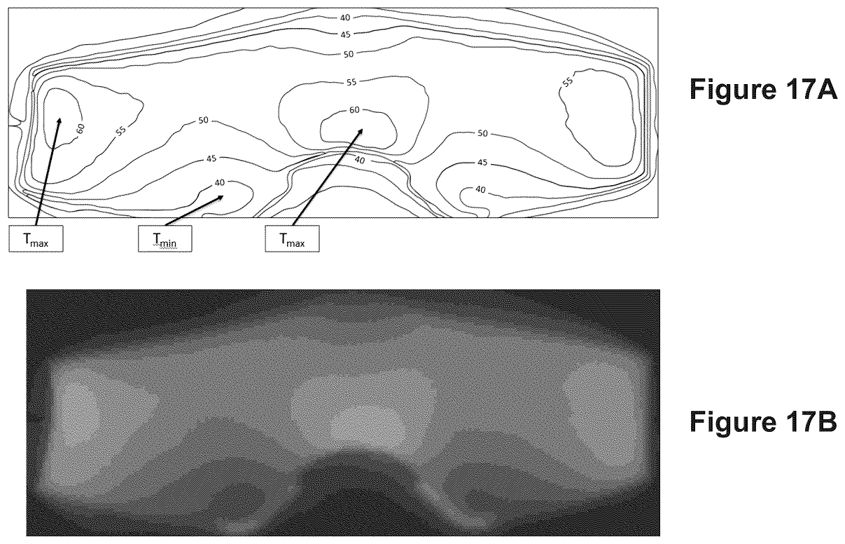

Similar results can be observed in FIGS. 17A and 17B, where a power input of 5.6 W, corresponding to about 64% of that of the prior art, was used. As noted in Table 1, while the temperatures are overall lower (e.g., when compared to FIGS. 16A and 16B), the temperature variations are lower, at about 26.9° C., and the average temperature is about the same as the prior art embodiment, at about 49.7° C. However, this embodiment provides an improved temperature distribution, at about 6.7° C., while also maintaining the optical zone heated at about 55-60° C., and keeping known cold spots above 40° C. The temperature proximate the electrodes 104 is at about 60° C., which is lower than the embodiments of FIG. 16A (at about 102.3° C.) and the prior art (at about 103.2° C.). It is thus noted that the performance of the electric goggles, operated at 36% less power when compared to the prior art, is improved using the heating assembly of the present disclosure.

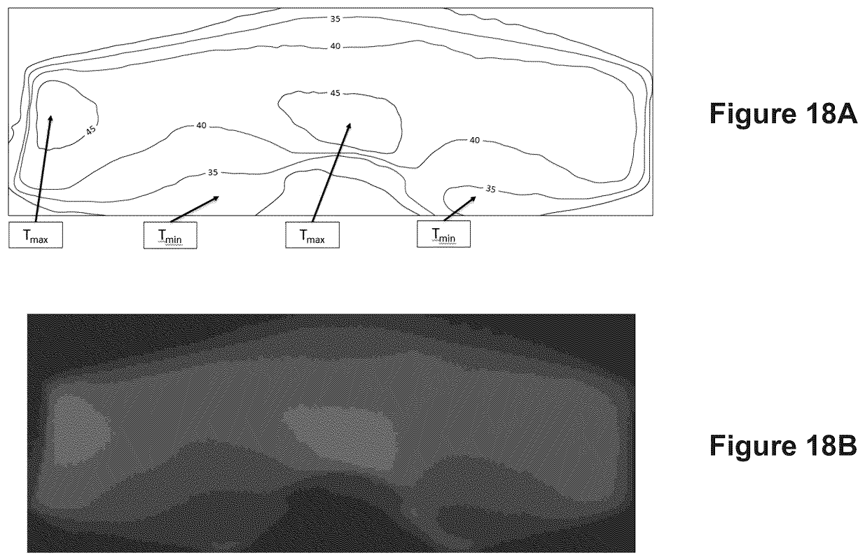

Yet other similar results are observed in FIGS. 18A and 18B, where a power input of 2.5 W, corresponding to about 28% of that of the prior art, was used. As noted in Table 1, while the temperatures are overall lower, the temperature variations are also lower, at about 16.7° C., with the average temperature being at about 40° C. (e.g., 39.3° C.). However, this embodiment provides an improved temperature distribution, at about 4.3° C., while also maintaining the optical zone heated at about 40-45° C., and keeping known cold spots around 35° C. The temperature proximate the electrodes 104 is at about 45° C., which is lower than all the other embodiments of Table 1. It is thus noted that the performance of the electric goggles, operated at 72% less power when compared to the prior art, is improved using the heating assembly of the present disclosure.

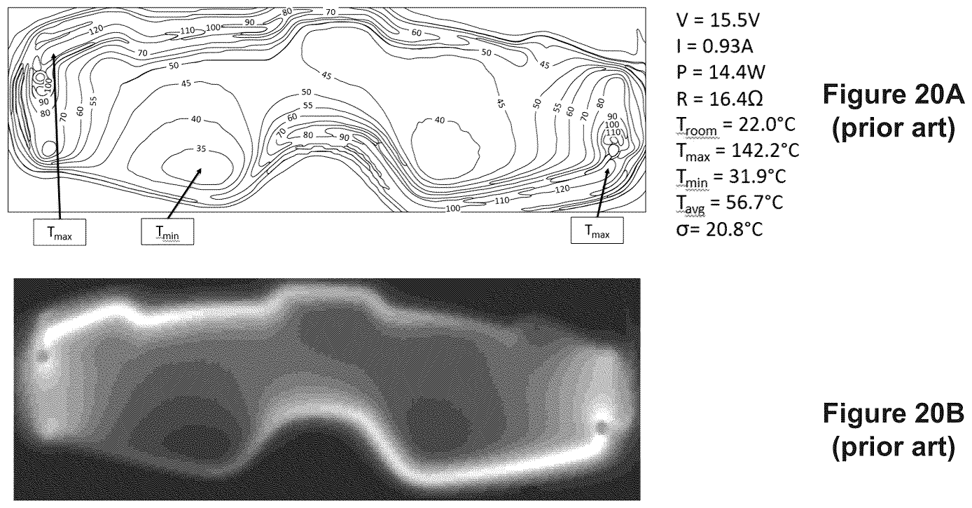

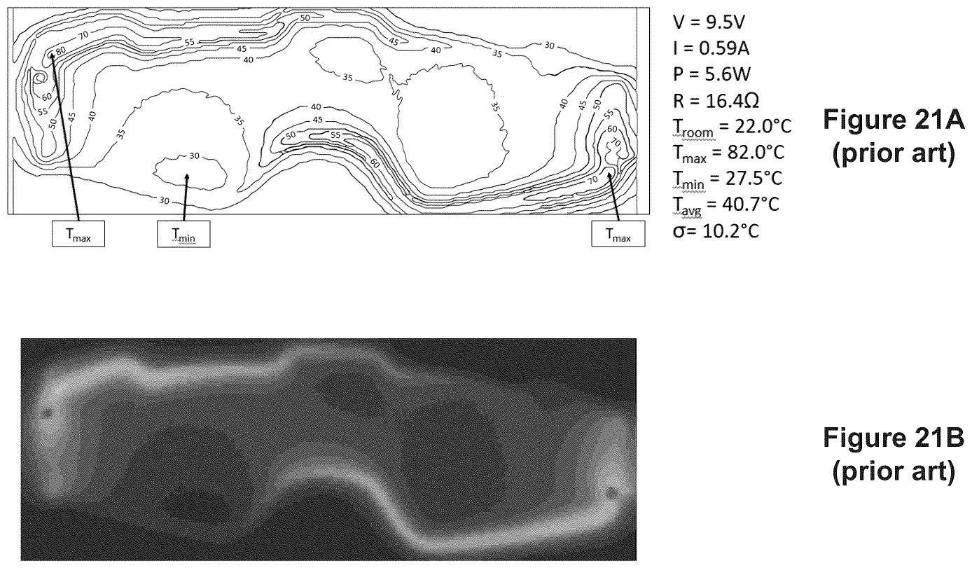

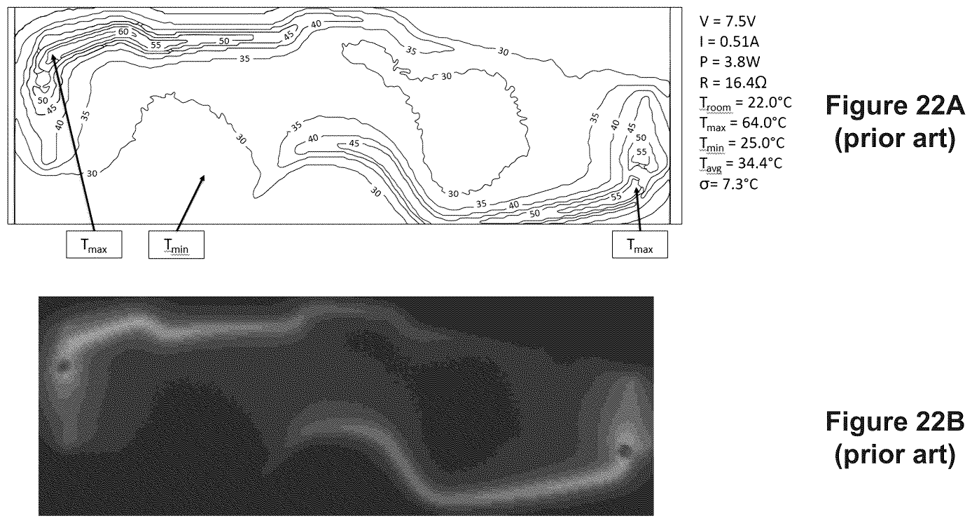

For comparison, FIGS. 20A to 22B illustrate the temperature distribution across the prior art's pair of goggles, which do not include lateral electrodes and instead include vertically-spaced electrodes, at different power inputs. FIGS. 20A and 20B show temperature distribution across the lens of the prior art with an input of 14.4 W. It is noted that, while the temperature distribution might be improved (e.g., when compared to FIGS. 19A and 19B), the lens is subjected to temperatures up to about 140° C., which can incur risks of injury to the user and/or overheating of the system, among others. FIGS. 21A to 22B show temperature distributions across the lens of the prior art with inputs of 5.6 W (FIGS. 21A and 21B) and 3.8 W (FIGS. 22A and 22B). As shown, some portions of the lens are not heated and remain around room temperature, indicative of poor performance of known heating systems at these levels of power. In order to better represent the improvement of the heated lens of the present disclosure, the coefficient of variation can be calculated for both the goggles described herein and the prior art goggles. Specifically, the coefficient of variation corresponds to a statistical measure of relative variability defined as the ratio of the standard deviation (σ) to the mean (μ), often expressed as a percentage (CV=(σ)/(μ)*100%). With reference to FIGS. 19 to 22, the CV are respectively 32%, 37%, 25% and 21%, whereas the CV of the goggles of the present disclosure corresponds to about 15%, representing an improvement over known technologies.

In some embodiments, the lens assembly 14 can be provided with one or more sensors 160 adapted to measure and monitor various parameters. For example, the sensors 160 can include a temperature sensor, a pressure sensor, a humidity sensor, a light/lumen sensor, a power sensor (e.g., to monitor a status and/or charge of the power source), etc. In some embodiments, the sensors 160 include PCB sensors printed onto a surface of the lens assembly, such as on the inner surface of the internal lens, for example. Alternatively, the sensors can be mounted to the lens assembly 14 via any suitable means. In yet other possible embodiments, one or more sensors can be provided on the frame 12, on the helmet or a combination thereof. The sensors 160 can be adapted to monitor respective parameters and programmed to activate the heating assembly when the parameters reach predetermined thresholds. For instance, the heating assembly can be activated upon detecting temperatures lower than a predetermined temperature threshold, or humidity levels higher than a predetermined humidity threshold. As such, the heating assembly can be automatically activated to prevent or reduce fog and/or frost accumulation on the lens assembly. In some embodiments, the sensors can be adapted to transmit wired and/or wireless signals to electronic components within the goggles (e.g., the controller) or to other devices, such as a smart phone or smart watch.

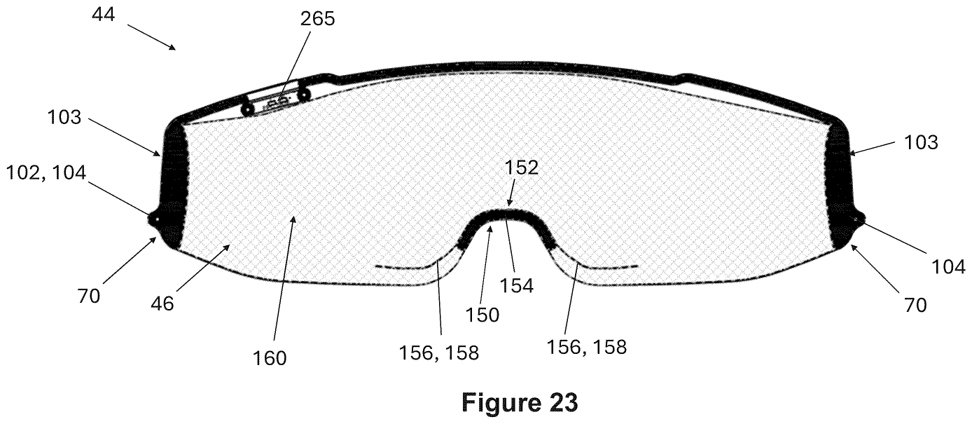

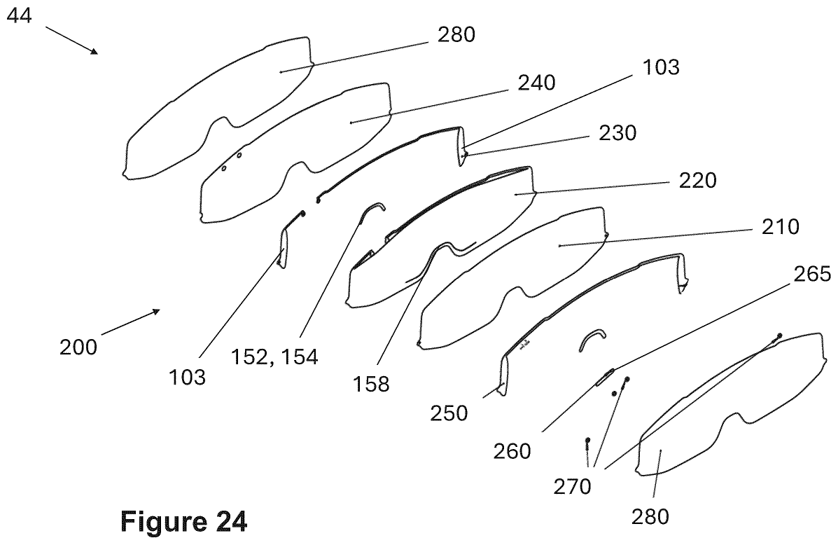

With reference to FIGS. 23 and 24, another embodiment of the internal lens 44 is shown. In this embodiment, the internal lens 44 has a stackup assembly 200 consisting of a plurality of layered components. For instance, the stackup assembly 200 includes a substrate or underlying layer 210 onto which other components can be connected to and/or applied on. The stackup assembly 200 also includes an isolated circuit layer 220 including the kiss cut 158, the silver ink components layer 230, including the bus bars 103 and the nose bridge electrical conductor 152 (e.g., the metallic component 154) and an antifog film 240. In this embodiment, the stackup assembly 200 includes an outline layer 250 generally matching the shape and configuration of the silver ink components layer 230. The stackup assembly 200 has a flexible circuit component 260 adapted to be positioned between two points in the electrical circuit. The flexible circuit component 260 includes an indicator 265, such as a LED, operable to indicate the status of the heating system (e.g., ON, OFF, etc.). Rivets 270 are also provided to define connection points for the electrical circuit, similar to previously described embodiments. Finally, the stackup assembly 200 includes a pair of protective layers 280 configured to “sandwich” the remaining components of the stackup assembly 200 therebetween. The protective layers 280 can be temporary, such as for improved protection during handling, packing, shipping, etc. Prior to use, the temporary protective layers 280 can be removed. Alternatively, the protective layers 280 can be permanent for improved protection of the internal lens 44.

It should be appreciated from the present disclosure that the various implementations of the electric goggles include a rigid main frame component (e.g., the frame body), a face-engaging component or inner component (e.g., the internal frame component) coupled on a first side of the rigid main frame component, and an environmental component or outer component (e.g., the lens assembly) coupled on a second side of the rigid main frame component, opposite the first side. It is thus noted that the goggles described herein can correspond to a three-part goggle assembly. However, in some embodiments, it is noted that each part of the “three-part goggle assembly” can be further broken down (e.g., separated) into additional sub-components. For example, the internal frame component can have a disconnectable foam overlay.

A plurality of features are integrated to the main frame component in order to improve the overall design of the goggles. For instance, the goggle ventilation and electronic components are integrated to the main frame component to enable removal and/or simple replacement of other components, such as the face-engaging component and/or the environmental component. The face-engaging component and/or the environmental component can be designed lighter, simpler and/or include generic components enabling cooperation and engagement with the main frame component. For example, the environmental component (e.g., the lens assembly) can include a flexible structure adapted to connect with the rigid structure of the main frame. The simpler and lighter design of these components can facilitate manufacturing, which in turn reduces the associated costs. It is appreciated that, by reducing the manufacturing costs of one or more components of the goggles, the goggles and/or each individual component can be sold at a lower price.

It should also be appreciated that the various implementations of the electric goggles are provided with a heating system having a transparent conductive film (TCF) of greater conductivity than known heating films. This improved conductivity allows for the electrodes (e.g., bus bars) to be positioned at opposite sides of the lens (i.e., on the left and right), instead of at the top and bottom. The TCF described herein also enables for less silver electrodes to be used and have reduced silver ink area, thereby reducing the overall manufacturing costs. The TCF is designed to also reduce the risks of overheating the electrodes and/or the lens surface proximate the electrodes. The resistivity of the TCF is typically more uniform than that of the prior art over the surface of the lens, and between separately produced lenses. In other words, the TCF can be manufactured and operated consistently and according to a repeatable process. The TCF provides gear light transparency and eliminates known oxidation/color change issues of other known heating films.

For example, and without being limitative, the TCF described herein has a surface resistivity of about 3 ohms/square, whereas known heating films typically have a surface resistivity of about 100 to 300 ohms/square in an acceptable transparency range. This lower surface resistivity enables the electrodes of the present disclosure to be spaced apart from one another laterally across the lens. This distance can increase the global resistivity of the heating system (e.g., of the lens) to about 10 ohms, “forcing” the electric current across the TCF generally uniformly. The lower resistivity of the TCF enables operation of the heating system with lower voltages while still providing sufficient heat to the lens. It should also be noted that, since the geometry of the lens does not create short circuits or paths of lower resistance, the voltage can still be increased (e.g., to 12V) without risking overheating of the system, the lens and/or the surrounding gear (e.g., helmet, jacket, strap, etc.).

Advantageously, the TCF includes a more rigid (or less brittle) assembly when compared to known heating films. For instance, the TCF is resilient to bending forces and is therefore less likely to be damaged while being handled during production and/or by the user.

The TCF, in combination with the copper micromesh, has an even greater electrical redundancy than the TCF without the copper micromesh. In other words, if a part of the lens is damaged, the damaged part will not interfere with standard operations of the lens (unless the damage/level of breakage is too great). For example, if a copper strand of the copper mesh breaks, the conductive bridge provided by the silver nanowires and/or carbon nanotubes maintains electrical continuity. Alternatively, the current will simply take another route through the copper mesh to avoid the damaged part. In stark contrast, with known heating films, breaking a part thereof would lead to other and/or greater issues such as breakage propagation across the film, the formation of major hot spots, melting of the film and loss of electrical continuity, for example. In addition, the carbon nanotubes are configured to improve abrasion and oxidation resistance of the cooperating elements (e.g., silver and/or copper). It is thus noted that the carbon nanotubes can have both mechanical and electrical protective roles.

Additionally, it should be noted that, as the temperature rises, the copper mesh, carbon nanotubes, and silver nanowires become less conductive. As such, the TCF components can provide a self-regulating or self-uniformizing structure with regards to the temperature, thereby avoiding overheating. Meanwhile, known technologies have been known to become more conductive as the temperature rises, which can cause an amplification or concentration effect on the current lines, leading to accentuation of hot spots. In other words, the heating system of the present disclosure has a stable and convergent state of equilibrium (i.e., the hotter it gets, the more uniform it is). Inversely, known heating technologies have an unstable state of equilibrium (i.e., the hotter it gets, the less uniform it is).

The present disclosure may be embodied in other specific forms without departing from the subject matter of the claims. The described example implementations are to be considered in all respects as being only illustrative and not restrictive. For example, the electric goggles can be provided with an activation button configured to control the duration and the intensity of the electrical current created by the heating assembly. The activation button can be located on the frame or coupled thereto (e.g., similar to the device 135) for ease of access and convenience. The goggles can have one or more activation modes operated by the activation button. For example, a first mode can be adapted to provide a constant amount of heat to the internal lens and a second mode can be adapted to provide an intense amount heat for a predetermined amount of time after which the heating assembly deactivates. The activation modes can be controlled via the sensors of the lens assembly. The sensors can be adapted to monitor humidity levels and/or temperatures within and/or around the goggles and can communicate with the controller to activate the heating assembly, for instance, when the humidity levels and/or the temperature reaches a predetermined threshold.

In other embodiments, the internal lens 44 can be directly laminated onto the external lens 42, thereby creating a lens assembly having a single heated lens. It should also be noted that the transparent conductive film (TCF) described herein can be replaced with known heating films, such as a conductive thin film compound which includes indium tin oxide, generally referred to as “ITO based films”. The ITO based film can be used and implemented in the goggles and the heating assembly as described herein. For instance, the ITO based film can be used with any one of the lateral electrodes, the electrical conductors, the kiss cut, the closed-loop electrical circuit, the outrigger structure, the power source, the controller, etc., or any suitable combination of the features described herein. However, it should be noted that the overall optics of the lens (e.g., the visible light transmission (VLT)) may be reduced.

While the electric goggles have been described in conjunction with the exemplary embodiments described above, many equivalent modifications and variations will be apparent to those skilled in the art when given this disclosure. Accordingly, the exemplary embodiments set forth above are considered to be illustrative and not limiting. The scope of the claims should not be limited by the preferred embodiments set forth in this disclosure, but should be given the broadest interpretation consistent with the description as a whole.

Moreover, although the embodiments of the goggles and corresponding parts thereof consist of certain geometrical configurations as explained and illustrated herein, not all of these components and geometries are essential and thus should not be taken in their restrictive sense. It is to be understood, as also apparent to a person skilled in the art, that other suitable components and cooperation thereinbetween, as well as other suitable geometrical configurations, may be used for the goggles, as will be briefly explained herein and as can be easily inferred herefrom by a person skilled in the art. Moreover, it will be appreciated that positional descriptions such as “above”, “below”, “left”, “right” and the like should, unless otherwise indicated, be taken in the context of the figures and should not be considered limiting.

The described example implementations are to be considered in all respects as being only illustrative and not restrictive. In the present disclosure, an implementation is an example or embodiment of the goggles. The various appearances of “one implementation,” “an implementation” or “some implementations” do not necessarily all refer to the same implementations. Although various features may be described in the context of a single implementation, the features may also be provided separately or in any suitable combination. Conversely, although the goggles may be described herein in the context of separate implementations for clarity, it may also be embodied in a single implementation. Reference in the specification to “some implementations”, “an implementation”, “one implementation”, or “other implementations”, means that a particular feature, structure, or characteristic described in connection with the implementations is included in at least some implementations, but not necessarily in all implementations.

As used herein, the terms “coupled”, “coupling”, “attached”, “connected” or variants thereof as used herein can have several different meanings depending in the context in which these terms are used. For example, the terms coupled, coupling, connected or attached can have a mechanical connotation. For example, as used herein, the terms coupled, coupling or attached can indicate that two elements or devices are directly connected to one another or connected to one another through one or more intermediate elements or devices via a mechanical element depending on the particular context.

Similarly, positional descriptions such as “top”, “bottom”, “above”, “under”, “below”, “left”, “right”, “front”, “rear”, “parallel”, “perpendicular”, “transverse”, “inner”, “outer”, “internal”, “external”, and the like should, unless otherwise indicated, be taken in the context of the figures and should not be considered limiting.

In the above description, the same numerical references refer to similar elements. Furthermore, for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The implementations, geometrical configurations, materials mentioned and/or dimensions shown in the figures are optional, and are given for exemplification purposes only.

In addition, although the optional configurations as illustrated in the accompanying drawings comprises various components and although the optional configurations of the frac plug as shown may consist of certain geometrical configurations as explained and illustrated herein, not all of these components and geometries are essential and thus should not be taken in their restrictive sense, i.e. should not be taken as to limit the scope of the present disclosure. It is to be understood that other suitable components and cooperations thereinbetween, as well as other suitable geometrical configurations may be used for the implementation and use of the goggles, and corresponding parts, as briefly explained and as can be easily inferred herefrom, without departing from the scope of the disclosure.

1. A pair of goggles to be worn in combination with a helmet to selectively cover a front opening of the helmet, the pair of goggles comprising:
a frame having a top frame edge, a bottom frame edge and opposite lateral frame edges together defining a closed perimeter and an opening;
a strap assembly comprising at least one strap clip coupled to the frame and a strap connectable to the strap clip, the strap being adapted to surround the helmet or be connected thereto;
a lens assembly removably connectable to the frame to cover the opening, the lens assembly comprising:
a lens frame having a top lens edge, a bottom lens edge and opposite lateral lens edges defining a lens opening; and
a lens connectable to the lens frame to cover the lens opening; and
a heating assembly configured to generate and provide heat to the lens assembly, the heating assembly comprising:
conductive elements provided on the lens and configured to enable an electrical current to travel therebetween, the conductive elements being provided on opposite sides of the lens such that, upon connecting the lens to the lens frame, the conductive elements are located proximate respective lateral lens edges of the lens frame.

2. A lens assembly for a pair of electric goggles provided with a heating assembly, the lens assembly comprising:
a lens frame having a top lens edge, a bottom lens edge and opposite lateral lens edges defining a lens opening; and
a lens connectable to the lens frame to cover the lens opening, the lens comprising conductive elements spaced across a surface of the lens and configured to enable an electrical current to travel therebetween, the conductive elements being provided on opposite sides of the lens such that, upon connecting the lens to the lens frame, the conductive elements are located proximate respective lateral lens edges of the lens frame.

3. A pair of electrical goggles, comprising:
a three-part assembly, comprising:
a rigid frame;
an inner component removably connected to a first side of the rigid frame;
an outer component removably connected to a second side of the rigid frame, opposite the first side; and
a heating assembly configured to generate and provide heat to at least one of the inner component and the outer component, the heating assembly comprising:
a pair of conductive elements provided on the at least one of the inner component and the outer component and configured to enable an electrical current to travel therebetween, the conductive elements being provided on opposite ends of the at least one of the inner component and the outer component such that the electrical current travels across a distance of the at least one of the inner component and the outer component, the distance corresponding to a greater one between a length and a height of the at least one of the inner component and the outer component.
